(19) United States
(12) Patent Application Publication
(10) Pub. No.: US 2013/0116206 A1
(43) Pub. Date: May 9, 2013

(54) QUINOLINE DERIVATIVES USED AS PET IMAGING AGENTS

(71) Applicants: Federica Pisaneschi, South Kensington (GB); Alan Spivey, South Kensington (GB); Graham Smith, South Kensington (GB); Eric Aboagye, South Kensington (GB)

(72) Inventors: Federica Pisaneschi, South Kensington (GB); Alan Spivey, South Kensington (GB); Graham Smith, South Kensington (GB); Eric Aboagye, South Kensington (GB)

(73) Assignee: IMPERIAL INNOVATIONS LIMITED, South Kensington, London, GB (GB)

(21) Appl. No.: 13/518,000

(22) PCT Filed: Dec. 22, 2010
(86) PCT No.: PCT/GB10/02325

§ 371 Date: Jan. 16, 2013

(30) Foreign Application Priority Data
Dec. 22, 2009 (GB) 0922302.5

(51) Int. Cl. C07D 215/54; C07H 19/056; A61K 31/706; A61K 31/4706; A61K 31/4709; C07D 401/12; A61K 45/06

(52) U.S. Cl. CPC C07D 215/54; C07D 401/12; C07H 19/056; A61K 45/06; A61K 31/4706; A61K 31/4709; A61K 31/706
USPC 514/43; 546/160; 536/28.7; 514/313

(57) ABSTRACT

There is provided compounds of formula (I), wherein R¹, R², X¹, X², and X³ have meanings given in the description, and pharmaceutically-acceptable salts thereof, which compounds are useful as positron emission tomography (PET) imaging agents, useful in the treatment of diseases in which inhibition of epidermal growth factor receptor tyrosine kinase activity or the inhibition of HER2 activity is desired and/or required, and useful in the treatment of cancer.

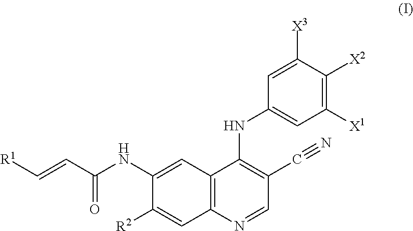

The present invention concerns compounds which have utility in therapeutic and diagnostic applications. In particular, the invention provides compounds which are useful as positron emission tomography (PET) imaging agents for assessing epidermal growth factor receptor (EGFR) status in vivo. The compounds are considered to be useful in prognosis and prediction of therapeutic response for various conditions. The compounds are also useful in treating or preventing diseases, such as cancer, in which inhibition of epidermal growth factor receptor kinase activity is desired and/or required.

EGFR, along with the other three members of the HER (human epidermal growth factor receptor) family (HER2, HER3 and HER4), is important in the carcinogenesis of the breast and in the therapeutic response of breast cancer (Marmor et al. (2004) Int. J. Radiat. Oncol. Biol. Phys. 58: 903-913; Bazley & Gullick (2005) Endoncr. Relat. Cancer 12: S17-S27). In addition to breast cancer, these receptors are overexpressed in other cancers including ovarian, endometrial and non-small cell lung cancer.

EGFR is a transmembrane glycoprotein that comprises an extracellular ligand-binding domain, a transmembrane domain and an intracellular domain with tyrosine kinase activity. Once activated by binding to a variety of ligands like EGF, amphiregulin and TGF-α, it is believed to undergo homo- or heterodimerisation with Her2 or other members of the family followed by activation of the intrinsic protein tyrosine kinase by autophosphorylation. The latter activates intracellular signal transduction pathways such as phosphatidylinositol-3-kinase (PI3K)/AKT and the ras/raf/MEK/MAPK pathways (Normanno et al (2002) J. Cell. Physiol. 194: 13-19; Salomon et al (1995) Crit. Rev. Oncol. Haematol. 19: 183-232; Woodburn (1999) Pharmacol. Ther. 82: 241-250).

The blockage of the activity of one or more members of the HER family by inhibiting their tyrosine kinase domains appears to be a valid anti-cancer strategy, therefore inhibitors of EGFR are preferred targets both as anti-cancer drugs and as imaging agents for PET.

Previous attempts to develop small molecule imaging agents have focused mainly on the replacement of radiohalogens in the original positions of compounds developed for therapy like Iressa (Seimbille at al (2005) J Label Compd Radiopharm 48: 829-843). They are all based on a 4-anilinoquinazoline core and are reversible inhibitors of EGFR. Although they have shown potential as radioimaging agents in vitro, this promise has not translated into high signal-to-noise PET images of EGFR-overexpressing tumours in animal models. For this class of reversible inhibitors, the failure could be attributed to a number of factors including high log P, rapid metabolism and blood clearance, and high (mM) intracellular levels of ATP that can compete with radiolabelled compound and lead to rapid cellular clearance.

It is thought that extremely high affinity compounds (low pM) or irreversible inhibitors of EGFR could overcome rapid cellular clearance attributable to high intracellular ATP content. Thus irreversible EGFR inhibitors have been exploited within the context of developing kinase-based imaging agents. This class is characterized by the presence of an electrophile on the C-6 position of the quinazoline core which binds covalently to a Cys 773 present in the tyrosine kinase binding site of EGFR. The covalent binding attenuates the ATP-induced washout and usually confers a higher potency (Yun et al (2008) PNAS. USA. 105: 2070-2075).

During the last few years, Mishani et al. have reported ¹¹C, ¹⁸F and ¹²⁴I anilinoquinazoline radiolabelled irreversible inhibitors (Ortu at al (2002) Int. J. Cancer. 101: 360-370; Abourbeh at al (2007) Nucl. Med. Biol. 34: 55-70) which showed a remarkable inhibitory effect in in vitro studies but did not perform well as PET imaging agents in vivo due to rapid metabolic degradation, low tumour uptake (Ortu et al (2002) Int. J. Cancer. 101: 360-370), and probably high non-specific uptake due to the high log P (Abourbeh et al (2007) Nucl. Med. Biol. 34: 55-70). Other inhibitors with lower logP have been synthesized by the same group and are currently under investigation (Dissoki at al (2007) Appl. Radiat. Isot. 65: 1140-1151).

Other investigators are exploiting radiolabelled antibodies to EGFR for imaging the target (nanobodies (Tijink at al (2008) Mol. Cancer. Ther. 8: 2288-2297), affibodies (Nordberg at al (2007) J. Nucl. Med. Blot 34: 609-618), full length+PEG (Wen et al (2001) J Nucl Med 42: 1530-1537), full length cetuximab (Ping et al (2008) Cancer Biother Radiopharm. 23: 158-71) and full length panitumumab (J Nucl Med. 2009 Jun. 12. [Epub ahead of print])). Whereas the full length antibodies have higher affinity, they also display slow kinetics, reduced tumour penetration and high liver uptake.

Compounds having a 3-cyano quinoline core have previously been reported (Torrance at al (2000) Nature Medicine 6: 1024-1028; Wssner et al (2003) J. Med. Chem. 46: 49-63; Tsou at al (2005) J. Med. Chem. 48: 1107-1131).

The listing or discussion of an apparently prior-published document in this specification should not necessarily be taken as an acknowledgement that the document is part of the state of the art or is common general knowledge.

The present inventors have designed and synthesized novel compounds based on the 3-cyanoquinoline core. Radiolabelled compounds of the invention can be used as irreversible EGFR imaging agents or HER2 imaging agents.

The present invention is concerned with compounds of formula I,

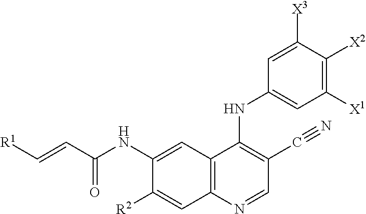

wherein:
R¹ represents Het^{a} or a C₁₋₃₀ alkyl group optionally substituted by one or more A groups;
R² represents a C₁₋₃₀ alkyl group optionally substituted by one or more B groups or one or more halogen atoms; a C₁₋₁₂-alkoxy group optionally substituted by one or more halogen atoms or hydroxyl groups; or Het^{b};
X¹ and X³ each independently represents hydrogen or a halogen;
A represents Het^{b}, —N(e)Ra², —OR^{a3} or —SR^{a4};
B represents —N(R^{b1})^{Rb2}, —OR^{b3} or —SR^{b4};
X² represents hydrogen, a halogen, OR^{c1}, SR^{c2}, Het^{d} or a C₁₀₀ alkyl group optionally substituted by one or more halogen atoms or one or more C groups;
C represents —N(R^{d1})R^{d2}, —OR^{d3} or —SR^{d4};
Het^{a} represents a heteroaryl group which may be optionally substituted by one or more halogen atoms or R^{d} groups;
Het^{b} represents a heteroaryl group which may be optionally substituted by one or more halogen atoms or R^{e} groups;
Het^{c} represents a heteroaryl group which may be optionally substituted by one or more halogen atoms or R^{f} groups;
R^{a1} to R^{a4}. R^{b1} to R^{b4} and R^{d1} to R^{d4} each independently represent hydrogen, a C(O)OR⁹ group, a C₁₋₆ alkyl group or a —C(O)—C₁₋₆ alkyl group, which latter two groups are optionally substituted with one or more D groups, one or more E groups and/or one or more halogen atoms;

R^{c1} and R^{c2} independently represent a C₁₋₁₂ alkyl group, a C₁₋₄-alkyl-C₃₋₈-cycloalkyl group, a C₁₋₄-alkyl-aryl group or a C₁₋₄-alkyl-Het^{d} group;

D represents an aryl group optionally substituted by one or more halogen atoms or R^{h} groups, or a Het^{e} group;
Het^{d} represents a heteroaryl group which may be optionally substituted by one or more halogen atoms or Rⁱ groups;
Het^{e} represents a heteroaryl group which may be optionally substituted by one or more halogen atoms or R^{j} groups;
E represents —O—N(R^{k})R^{l} or —O—N═C(R^{m})Rⁿ;
R^{d}, R^{e}, R^{f}, R^{g}, R^{h}, Rⁱ and R^{j} independently represent:
a C₁₋₆ alkyl group optionally substituted by one or more halogen atoms or another suitable leaving group (e.g. a p-toluenesulfonate (Ts), a methanesulfonate (Ms), a p-nitrobenzenesulfonate (4-Ns), an o-nitrobenzenesulfonate (2-Ns), or a trifluoromethanesulfonate (Tf) group); or
a Q group

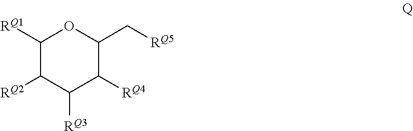

wherein one of R^{Q1} to R^{Q5} represents the point of attachment to the quinoline-containing portion of the molecule, one or more of R^{Q1} to R^{Q5} represents a halogen atom or another suitable leaving group (e.g. a p-toluenesulfonate, a methanesulfonate, a p-nitrobenzenesulfonate, an o-nitrobenzenesulfonate or a trifluoromethanesulfonate group), and the remaining R^{Q1} to R^{Q5} groups represent —OH;
   R^{k}, R^{l}, R^{m} and Rⁿ each independently represent hydrogen or a C₁₋₁₂ alkyl group optionally substituted by one or more halogen atoms, —OR^{o} or —N(R^{p})R^{q} groups;
   R^{o}, R^{p} and R^{q} each independently represent hydrogen or a C₁₋₄ alkyl group;
   or a pharmaceutically-acceptable salt thereof.

In a particular aspect, the present invention provides compounds of formula I as defined above provided that:

(i) when X³ represents hydrogen, X² represents fluoro, and X¹ represents chloro,
(a) when R² represents —O—CH₂CH₃, R¹ does not represent —CH₂—N(CH₃)₂ or —CH₂—N(H)CH₃;
(b) when R² represents —O—CH₃, R¹ does not represent —CH₂—N(CH₃)₂, —CH₂—N(CH₂CH₃)₂, —CH(CH₃)—N(CH₃)₂ and —CH(CH₃)—N(CH₂CH₃)₂;
(c) when R² represents —O—CF₃, R¹ does not represent —CH₂—N(CH₃)₂;
   (ii) when X² and X³ represent hydrogen, X′ represents bromo, and R¹ represents —CH₂—N(CH₃)₂, R² does not represent —O—CH₃ or —O—CH₂CH₃;
   (iii) when X¹ represents chloro, X³ represent hydrogen, R¹ represents —CH₂—N(CH₃)₂ and R² represents —O—CH₃, X² does not represent imidazol-1-yl; and
   (iv) when R² represents —O—CH₂CH₃ or —O—CH₃, X¹ represents hydrogen or chlorine, X³ represents hydrogen or chlorine and X² represents OR^{c1}, the compound contains at least one fluorine atom.

The compounds of formula I (both all of the compounds of formula I and formula I when limited by the provisos) and their salts are referred to hereinafter as “the compounds of the invention”. The comments below relating to the compounds of the invention and their uses apply to all compounds within the definition of formula I. It should also be understood that in a particular aspect of the invention compounds of formula I, as restricted by the provisos, are used in the applications, uses, formulations etc discussed below.

Pharmaceutically-acceptable salts include acid addition salts and base addition salts. Such salts may be formed by conventional means, for example by reaction of a free acid or a free base form of a compound of formula I with one or more equivalents of an it) appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

The term “halogen”, when used herein, includes fluoro, chloro, bromo and iodo.

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. Other aryl groups which may be mentioned include those where the rings are directly linked but not fused, e.g. biphenyl. The point of attachment of aryl groups may be via any atom of the ring system. However, when aryl groups are bicyclic or tricyclic, they are linked to the rest of the molecule via an aromatic ring.

The heteroaryl groups in compounds of formula I that may be mentioned (i.e. heteroaryl groups which are represented by Het^{a}, Het^{b}, Het^{c}, Het^{d} and Het^{e}) include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroatoms that may be mentioned include phosphorus, silicon, boron, tellurium, selenium and, preferably, oxygen, nitrogen and sulphur. Heteroaryl groups may also be fused to other aryl or heteroaryl groups. Heterocyclic groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl) and, more preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazopyridyl (such as imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Preferred heteroaryl groups include pyrrole, pyrazole, imidazole, 1,2,3-triazole, 1,2,4-triazole, furan, oxazole, isoxazole, thiophene, thiazole isothiazole, 2-pyridine, 3-pyridine, and 4-pyridine which groups may be optionally substituted by one or more R^{d}, R^{e}, R^{h}, Rⁱ or R^{j} groups as appropriate.

More preferred heteroaryl groups include 1,2,3-triazole and 2-pyridine, which groups may be optionally substituted by one or more R^{d}, R^{e}, R^{h}, Rⁱ or R^{j} groups as appropriate.

For the avoidance of doubt, in cases in which the identity of two or more substituents in a compound of the invention may be the same, the actual identities of the respective substituents are not in any way interdependent. For example, in the situation in which X¹ and X² both represent a halogen, the halogens in question may be the same or different.

For the avoidance of doubt, when a term such as “R^{a1} to R^{a4}” is employed herein, this will be understood by the skilled person to mean R^{a1}, R^{a2}, R^{a3} and R^{a4} inclusively.

The invention disclosed herein also encompasses all pharmaceutically acceptable compounds of the invention including those isotopically-labelled by having one or more atoms replaced by an atom having a different atomic mass or mass number. Examples of isotopes that can be incorporated into the compounds of the invention include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively.

These radiolabelled compounds could be useful to help determine or measure the effectiveness of the compounds. Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁵O, ¹³N and, particularly, ¹⁸F can be useful in Positron Emission Tomography (PET) studies. Isotopically-labelled compounds of the invention can generally be prepared by conventional techniques known to those skilled in the art or by processes analogous to those described in the Examples and Preparations as set out below using an appropriate isotopically-labelled reagent in place of the non-labelled reagent previously employed.

Compounds of the invention that may be mentioned include those in which:

R^{d}, R^{e}, R^{f}, R^{g}, R^{h}. Rⁱ and R^{j} independently represent a C₁₋₆ alkyl group optionally substituted by one or more halogen atoms or another suitable leaving group (e.g. a p-toluenesulfonate (Ts), a methanesulfonate (Ms), a p-nitrobenzenesulfonate (4-Ns), an o-nitrobenzenesulfonate (2-Ns), or a trifluoromethanesulfonate (Tf) group); and/or R² represents a C₁₋₃₀ alkyl group optionally substituted by one or more B groups or one or more halogen atoms; a C₁₋₁₂-alkoxy group optionally substituted by one or more halogen atoms; or Het^{b};

Compounds of the invention that may be mentioned include those in which:

R¹ represents Het^{a} or a C₁₋₆ alkyl group optionally substituted by one or more A groups, wherein A preferably represents —N(R^{a1})R^{a2}; and/or
R² represents a C₁₋₆ alkyl group optionally substituted by one or more B groups, a C₁₋₆-alkoxy group optionally substituted by one or more halogen atoms, or Het^{b}; and/or at least one of X¹ and X³ represents hydrogen.

Further compounds of the invention that may be mentioned include those in which:

R¹ represents Het^{a} or a C₁₋₆ alkyl group optionally substituted by one or more A groups, wherein A preferably represents —N(R^{a1})R^{a2}; and/or
R² represents a C₁₋₆-alkoxy group optionally substituted by one or more halogen atoms, a C₁₋₆ alkyl group optionally substituted by one or more halogen atoms, or Het^{b}; and/or
X² represents a halogen (e.g. fluorine), OR^{c1} or SR^{c2}; wherein if present
Het^{a} represents a heteroaryl group which may be optionally substituted by one or more R^{d} groups; and/or
Het^{b} represents a heteroaryl group which may be optionally substituted by one or more R^{e} groups; and/or
Het^{c} represents a heteroaryl group which may be optionally substituted by one or more R^{h} groups; and/or
Het^{d} represents a heteroaryl group which may be optionally substituted by one or more Rⁱ groups; and/or
Het^{e} represents a heteroaryl group which may be optionally substituted by one or more R^{j} groups; and/or
R^{o}, R^{p} and R^{q} all represent hydrogen.

Preferred compounds of the invention include those in which:

R¹ represents Het^{a} or a C₁₋₆ alkyl group optionally substituted by one or more A groups,
wherein A preferably represents —N(R^{al})R^{a2}; and/or
R² represents a C₁₋₂-alkoxy group optionally substituted by one or more halogen atoms (e.g. fluorine), a C₁₋₆ alkyl group optionally substituted by one or more halogen atoms, or Het^{b}; wherein if present
D represents either an aryl group optionally substituted by one or more halogen atoms (e.g. fluorine), or a heteroaryl group optionally substituted by one or more R^{j} groups; and/or
E represents —O—NH₂ or —O—N═CHR^{m}; and/or
R^{k}, R^{l}, R^{m} and Rⁿ each independently represent hydrogen or a C₁₋₁₂ alkyl group optionally substituted by one or more halogen atoms or —OH; and/or
R^{d}, R^{e}, R^{f}, R^{g}, R^{h}, Rⁱ and R^{j} independently represent a C₁₋₆ alkyl group optionally substituted by one or more halogen atoms (e.g. fluorine), a p-toluenesulfonate group, a methanesulfonate group, a trifluoromethanesulfonate, a p-nitrobenzenesulfonate or an o-nitrobenzenesulfonate group; and/or
R^{c1} represents a cyclohexylmethyl group, a pyridinylmethyl group or a triazolylmethyl group, which latter group is optionally substituted by one or more halogen atoms or R^{h} groups.

Further preferred compounds of the invention include those in which:

Het^{a}, Het^{b} Het^{c}, Het^{d} and Het^{e} each independently represents 1,2,3-triazole or 2-pyridine which groups may be optionally substituted by one or more R^{d}, R^{e}, R^{h}, Rⁱ or R^{j} groups respectively; and/or
R^{m} represents a C₁₋₁₂ alkyl group optionally substituted by one or more halogen atoms or —OH; and/or
R^{d}, R^{e}, R^{h}, Rⁱ and R^{j} each independently represent a C₁₋₂ alkyl optionally substituted with a halogen (e.g. fluorine).

Preferred compounds of the invention include those in which:

R¹ represents

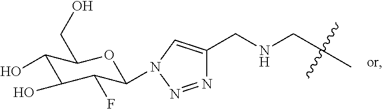 [SMILES: *CNCc1cn(C2OC(CO)C(O)C(O)C2F)nn1]

particularly, —CH₂N(CH₃)CH₂CH₂F, —CH₂N(CH₃)CH₂C₆H₄F, —CH₂NH(CH₃), —CH₂NHCH₂C≡CH, —CH₂N(boc)CH₂C≡CH, —C≡CH, —CH₂NHCH₂CH₂ONH₂, —CH₂NHC(O)CH₂ONH₂,

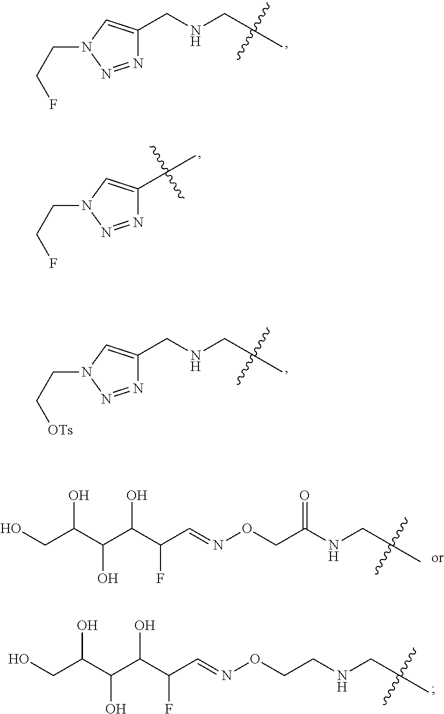 [SMILES: *CNCc1cn(CCF)nn1 ; *c1cn(CCF)nn1 ; *CNCc1cn(CCOS(=O)(=O)c2ccc(C)cc2)nn1 ; *CNC(=O)CO/N=C/C(F)C(O)C(O)C(O)CO ; *CNCCO/N=C/C(F)C(O)C(O)C(O)CO]

and/or
R² represents —OCH₂CH(OH)CH₂(OH) or, particularly, —CH₂(CH₂)ₘ—F, —(CH₂)ₙCH═CH₂ or preferably —OCH₂CH₃, —OCH₂CH₂F, —C≡CH, or

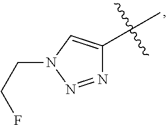

wherein m represents from 0 to 29 and n represents from 0 to 28.

Compounds of interest include those in which:

R¹ represents Het^{a} or a C₁₋₆ alkyl group optionally substituted by one or more A groups, wherein A preferably represents —N(R^{a1})R^{a2}; and/or
R² represents a C₁₋₆ alkyl group optionally substituted by one or more B groups, a C₁₋₆-alkoxy group optionally substituted by one or more halogen atoms, or Het^{b}; and/or
X¹ and X³ independently represents hydrogen or halogen (e.g. chlorine) (for example X¹ represents hydrogen and X³ represents halogen) and X² represents halogen (e.g. fluorine),

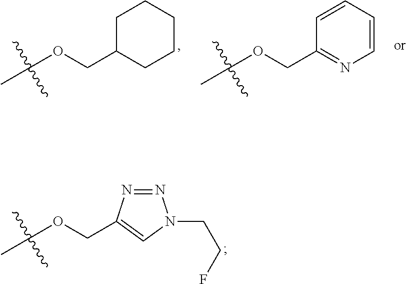 [SMILES: *OCC1CCCCC1 ; *OCc1ccccn1 ; *OCc1cn(CCF)nn1]

and/or
X² represents hydrogen, OR^{c1} or SR^{c2}; and/or
R^{a1} to R^{a4} each independently represent hydrogen, a C(O)OR^{f} group, a C₁₋₆ alkyl group which is optionally substituted with one or more D groups and/or one or more halogen atoms, or a —C(O)—C₁₋₆ alkyl which is optionally substituted with one or more E groups and/or one or more halogen atoms.

Compounds of interest include those in which:

R¹ represents

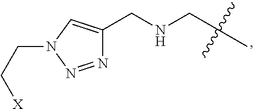

wherein X represents a substituent selected from p-toluenesulfonate, methanesulfonate, p-nitrobenzenesulfonate, o-nitrobenzenesulfonate, trifluoromethansulfonate, fluoro, chloro, bromo or iodo.

Compounds of interest include those in which R² represents —O—CH₂CH₃ or —O—CH₃, X¹ represents hydrogen or chlorine, X³ represents hydrogen or chlorine and X² represents OR^{c1}, and R¹, R² or X² contains fluorine.

Compounds of interest include those in which, when R² represents —O—CH₂CH₃ or —O—CH₃: X¹ represents hydrogen or chlorine, X³ represents hydrogen or chlorine and X² represents OR^{c1}, at least one of R¹ and R^{c1} contains a 1-(2-fluoro-ethyl)-1H-[1,2,3]triazol-4-yl moiety.

It should be understood that the disclosure of particular compounds of the invention in the previous nine paragraphs is a disclosure of these both not limited by the provisos and limited by the provisos.

Preferred compounds of formula I include:
{(E)-3-[4-(3-Chloro-4-fluoro-phenylamino)-3-cyano-7-ethoxy-quinolin-6-ylcarbamoyl]-allyl}-prop-2-ynyl-carbamic acid tert-butyl ester;
(E)-Pent-2-en-4-ynoic acid [4-(3-chloro-4-fluorophenylamino)-3-cyano-7-ethoxy-quinolin-6-yl]-amide;
(E)-4-[(2-Fluoroethyl)methyl amino]-but-2-enoic acid [4-(3-chloro-4-fluorophenylamino)-3-cyano-7-ethoxyquinoline-6-yl]amide;
(E)-4-[(4-Fluorobenzyl)methylamino]-but-2-enoic acid [4-(3-chloro-4-fluorophenylamino)-3-cyano-7-ethoxyquinoline-6-yl]amide;
(E)-4-{[1-(2-Fluoro-ethyl)-1H-[1,2,3]triazol-4-ylmethyl]-amino}-but-2-enoic acid [4-(3-chloro-4-fluoro-phenylamino)-3-cyano-7-ethoxy-quinolin-6-yl]-amide hydrochloride;
(E)-N-[4-(3-Chloro-4-fluorophenylamino)-3-cyano-7-ethoxyquinolin-6-yl]-3-[1-(2-fluoroethyl)-1H-[1,2,3]triazol-4-yl]-acrylamide;
(E)-4-Methylamino-but-2-enoic acid [4-(3-chloro-4-fluorophenylamino)-3-cyano-7-(2-fluoroethoxy)-quinolin-6-yl]-amide hydrochloride;
(E)-4-Prop-2-ynylaminobut-2-enoic acid [4-(3-chloro-4-fluorophenylamino)-3-cyano-7-ethoxy-quinolin-6-yl]-amide hydrochloride;
(E)-4-Methylamino-but-2-enoic acid {4-(3-chloro-4-fluoro-phenylamino)-3-cyano-7-[1-(2-fluoro-ethyl)-1H-[1,2,3]triazol-4-yl]-quinolin-6-yl}-amide;
Toluene-4-sulfonic acid 2-[4-({(E)-3-[4-(3-chloro-4-fluoro-phenylamino)-3-cyano-7-ethoxy-quinolin-6-ylcarbamoyl]-allylamino}-methyl)-[1,2,3]triazol-1-yl]-ethyl ester;
(E)-4-{[1-(2-Fluoro-ethyl)-1H-[1,2,3]triazol-4-ylmethyl]-amino}-but-2-enoic acid [4-(3-chloro-4-(cyclohexylmethoxy)-phenylamino)-3-cyano-7-ethoxy-quinolin-6-yl]-amide;
(E)-4-{[1-(2-Fluoro-ethyl)-1H-[1,2,3]triazol-4-ylmethyl]-amino}-but-2-enoic acid [4-(3-chloro-4-((pyridin-2-yl)methoxy)-phenylamino)-3-cyano-7-ethoxy-quinolin-6-yl]-amide;
(E)-4-{Methylamino}-but-2-enoic acid [4-(3-chloro-4-((1-(2-fluoro-ethyl)-1H-[1,2,3]triazol-4-yl)methoxy)-phenylamino)-3-cyano-7-ethoxy-quinolin-6-yl]-amide;
(E)-4-{2-(Aminooxy)-ethylamino}-but-2-enoic acid [4-(3-chloro-4-fluoro-phenylamino)-3-cyano-7-ethoxy-quinolin-6-yl]-amide;
(E)-4-{2-[2-Fluoro-3,4,5,6-tetrahydroxy-hex-(E)-ylideneaminooxy]-ethylamino}-but-2-enoic acid [4-(3-chloro-4-fluoro-phenylamino)-3-cyano-7-ethoxy-quinolin-6-yl]-amide;
(E)-4-{2-[2-Fluoro-3,4,5,6-tetrahydroxy-hex-(E)-ylideneaminooxy]-acetylamino}-but-2-enoic acid [4-(3-chloro-4-fluoro-phenylamino)-3-cyano-7-ethoxy-quinolin-6-yl]-amide;
(E)-4-{[1-(3-Fluoro-4,5-dihydroxy-6-hydroxymethyl-tetrahydropyran-2-yl)-1H-[1,2,3]triazol-4-ylmethyl]-amino}but-2-enoic acid [4-(3-chloro-4-fluoro-phenylamino)-3-cyano-7-ethoxyquinolin-6-yl]-amide; and
(E)-4-[(2-Fluoroethyl)-methyl-amino]-but-2-enoic acid [4-(3-chloro-4-fluorophenylamino)-3-cyano-7-(2,3-dihydroxypropoxy)-quinolin-6-yl]-amide.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which R² represents a C₁₋₁₂-alkoxy group substituted by one or more halogen atoms, reaction of a compound of formula II,

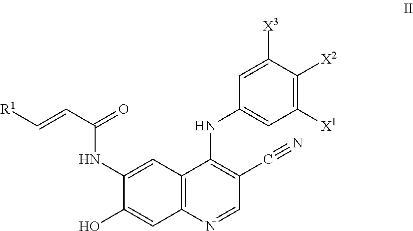

or a protected (e.g. at one of the amino groups) derivative thereof, wherein R¹, X¹, X² and X³ are as hereinbefore defined with a compound of formula III,

R^{2a}-L¹ III

wherein R^{2a} represents the optionally substituted C₁₋₁₂ alkyl portion of R², and L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). This reaction may be carried out at room temperature or above (e.g. at a high temperature, such as the reflux temperature of the solvent system that is employed) or using microwave irradiation; or
(ii) for compounds of formula I in which R¹ represents an optionally substituted 1,2,3-triazole group, reaction of a compound of formula I in which R¹ represents HC═C—, i.e. a compound of formula IV,

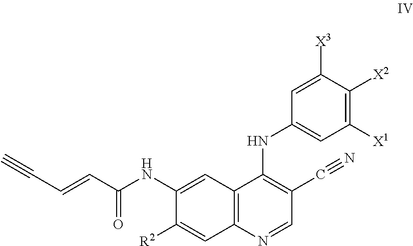

wherein R², X¹, X² and X³ are as hereinbefore defined, with a compound of formula V,

R^{1d}—N₃ V

wherein R^{1d} represents H or R^{d} as hereinbefore defined, under conditions known to those skilled in the art, for example in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃, Binol₂Ti₂O(O-i-pr)₂ or AgOAc and an optional additive such as Ph₃P, 2,2′-bis(diphenylphosphino)-1,1′-binaphthyl or xantphos, in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). This reaction may be carried out at room temperature or above (e.g. at a high temperature, such as the reflux temperature of the solvent system that is employed) or using microwave irradiation; or
(iii) reaction of a compound of formula VI,

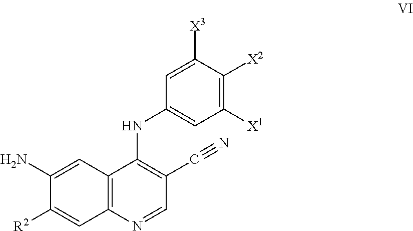

wherein R², X¹, X² and X³ are as hereinbefore defined, with a compound of formula VII,

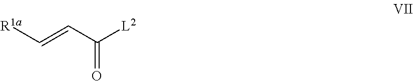

wherein R^{1a} represents R¹ as hereinbefore defined, and L² represents a suitable leaving group, for example a halogen, —OH or a C₁₋₆ alkoxy group, under standard coupling reaction conditions, for example (e.g. when L² represents —OH, or a C₁₋₆ alkoxy group) in the presence of a suitable coupling reagent (e.g. Al(CH₃)₃, 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluoro-phosphate, bromo-tris-pyrrolidinophosphonium hexafluorophosphate, 2-(1H-benzotriazol-1-yl)-1,1,3,3-tetramethyluronium tetrafluorocarbonate, 1-cyclohexyl-carbodiimide-3-propyloxymethyl polystyrene, O-(7-azabenzotriazol-1-yl)-N,N,N″,N″-tetramethyluronium hexafluorophosphate and/or O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof), an appropriate solvent (e.g. tetrahydrofuran, pyridine, toluene, dichloromethane, chloroform, acetonitrile, dimethylformamide, trifluoromethylbenzene, dioxane or triethylamine) and a further additive (e.g. 1-hydroxybenzotriazole hydrate). Alternatively, when L² represents certain leaving groups, e.g. chloro, such compounds may be prepared by converting the carboxylic acid group under standard conditions to the corresponding acyl chloride, e.g. in the presence of SOCl₂ or oxalyl chloride, prior to reacting the acyl chloride with a compound of formula VIII under similar conditions to those mentioned above; or
(iv) for compounds in which R¹ represents a C₁₋₃₀ alkyl group substituted by one or more —N(R^{a1})R^{a2} groups wherein at least one of R^{a1} and R^{a2} is a —CH₂—R^{ax} group wherein R^{ax} represents a D group, an E group, a halogen or a C₁₋₅ alkyl group optionally substituted with one or more D groups, one or more E groups and/or one or more halogen atoms, reaction of a compound of formula VIII,

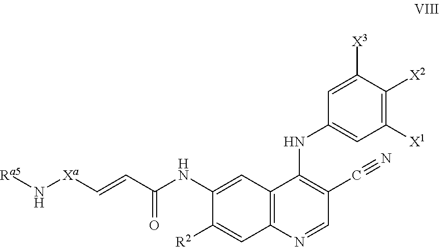

wherein R², X¹, X² and X³ are as hereinbefore defined, R^{a5} represents either R^{a1} or R^{a2}, and X^{a} represents the optionally substituted C₁₋₃₀ alkyl group of R¹, with a compound of formula IX,

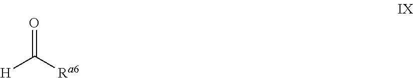

wherein R^{a6} represents R^{ax} as hereinbefore defined, followed by reduction of the resulting imine for example in the presence of a suitable reducing reagent such as LiAlH₄, NaBH₄ or trialkylsilane (e.g. triethylsilane) or reduction by hydrogenation (e.g. in the presence of Pd/C); or
(v) for compounds of formula I in which R¹ represents a C₁₋₃₀ alkyl group optionally substituted by —N(R^{a1})R^{a2}, reaction of a compound of formula X,

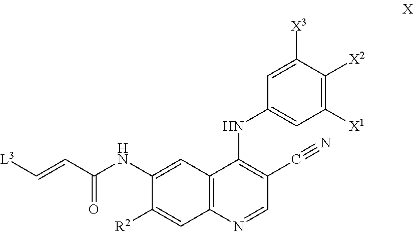

wherein R², X¹, X² and X³ are as hereinbefore defined, and L³ represents a suitable leaving group (such as chloro, bromo, iodo, a sulfonate group (e.g. —OS(O)₂CF₃, —OS(O)₂CH₃, —OS(O)₂PhMe or a nonaflate), —B(OH)₂ (or a protected derivative thereof, e.g. an alkyl protected derivative, so forming, for example a 4,4,5,5-tetramethyl-1,3,2-dioxaborolan-2-yl group), —Sn(alkyl)₃ (e.g. —SnMe₃ or —SnBu₃)) or a similar group known to the skilled person, with a compound of formula XI,

NH(R^{a1′})R^{a2′} XI

wherein R^{a1′} and R^{a2′} represent R^{a1} and Ra^{a2} as hereinbefore defined, respectively, under reaction conditions known to those skilled in the art, for example such as those described in respect of process step (i) above. The skilled person will appreciate that various groups (e.g. primary amino groups) may need to be mono-protected and then subsequently deprotected following reaction with the compound of formula X; or
(vi) for compounds of formula I wherein one or more of R^{d}, R^{e}, R^{f}, R^{g}, R^{h}, Rⁱ and R^{j} represents a C₁₋₆ alkyl group substituted by one or more halogen atoms, reaction of a compound of formula I wherein the corresponding R^{d}, R^{e}, R^{f}, R^{g}, R^{h}, Rⁱ or R^{j} group represents a C₁₋₆ alkyl group substituted by one or more leaving groups (e.g. a p-toluenesulfonate, a methanesulfonate, a p-nitrobenzenesulfonate, an o-nitrobenzenesulfonate or a trifluoromethansulfonate group), with an appropriate metal halide (e.g. KF), optionally in the presence of an appropriate crown ether, such as 18-crown-6-benzene, or a cryptand, such as 1,10-diaza-4,7,13,16,21,24-hexaoxabicyclo[8.8.8]hexacosane, in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). This reaction may be carried out at room temperature or above (e.g. at a high temperature, such as the reflux temperature of the solvent system that is employed) or using microwave irradiation.

Compounds of formulae II, III, IV, V, VI, VII, VIII, IX, X and XI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X¹, X², X³, R¹ and R² in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

The compounds of the invention can be used as nuclear imaging agents for detection of cancers that express the epidermal growth factor receptor. The use of the compounds of the invention in this way can overcome one or more limitations of existing agents. Such limitations include the use of carbon-11 in most of the cases or very complex radiochemical syntheses when fluorine-18 has been used.

The compounds of the invention are based on a 3-cyano quinoline core. Without wishing to be bound by theory, the inventors believe that these compounds show higher binding than previously explored compounds containing a quinazoline core.

It is thought that the introduction of the fluorine atom on the Michael acceptor is unlikely to affect binding (see Example 4, compounds 13 and 14 and Example 5, compound 17). The introduction of a fluorine atom on the C-7 (see Example 7, compound 24) and replacement of the C—O bond with a C—C bond is believed to increase the metabolic stability.

The compounds of the invention can give important information on structure activity relationship and metabolism when one uses these compounds as imaging agents. The compounds of the invention also have potential as anticancer drugs.

Late stage introduction of fluorine-18 (or other short-lived radioisotopes), by means of a convenient and efficient methodology (i.e. click chemistry), can be advantageous due to fewer reaction steps and hence higher radiochemical yields. Such a simplified process could be attractive for application to an automated radiosynthesis platform (e.g. GE FastLab™).

Compounds of formula I, as defined above, when not limited by the provisos, which contain a suitable radioisotope (such as ¹⁸F), may be used as nuclear imaging agents for detection of cancers that express the epidermal growth factor receptor.

No PET imaging agents based on a 3-cyano quinoline core have previously been reported.

The compounds of the invention provide multiple advantages over PET imaging agents that have been described previously. Such advantages include one or more of:

1. Long half-life due to the fluorine-18 isotope. Ability to use at sites that lack an on-site cyclotron.
2. Improved metabolic stability due to the fluorine containing moiety, use of secondary amines and C—C bond.
3. Ease of synthesis using click chemistry or other suitable approach in a permissive position on the Michael acceptor or on the C-7 groups, both not directly involved in receptor binding.
4. Irreversible functionality of the Michael acceptor leading to improved signal-to-noise ratio.
5. Rapid tissue penetration of small molecule enabling imaging within minutes to hours after injection of the radiolabelled compound.

In a particular aspect, the compounds of the invention comprise at least one fluorine-18. Preferably, the fluorine-18 is on the Michael acceptor, e.g. C-7.

The compounds of the invention (that is the compounds within the scope of formula I, including the compounds defined in the provisos) can be used as positron emission tomography (PET) imaging agents that could be used for the measure of the epidermal growth factor receptor (EGFR) status in vivo. Such probes could find utility in prognosis and prediction of therapeutic response including:

Detection of the transition of pre-malignant to malignant disease e.g. Barrett's oesophagus to oesophageal cancer.

Selection of patients with high EGFR expression e.g. in lung and breast cancer, and who may benefit from targeted therapies. Other proteins such as Ras may be important in the overall drug response.

Potentially, detection of EGFR mutations permitting patients to be prescribed alternative 2^{nd} line therapies.

Prediction of drug resistance, e.g. after overexpression after radiotherapy, or to endocrine therapy.

Monitor pharmacodynamic effects of a number of ATP competitive inhibitors of EGFR kinase e.g., in breast cancer.

Compounds of the invention, in particular compounds in which X² represents an —OR^{c1} or an —SR^{c2} group, can be used as positron emission tomography (PET) imaging agents that could be used to measure the statuses of other human epidermal growth factor receptors, particularly the HER2 receptor, in vivo. Preferred compounds which may be used as positron emission tomography (PET) imaging agents that could be used to measure the status of the HER2 receptor in vivo include compounds of formula I in which —OR^{c1} represents a (cyclohexyl)methoxy group, a (pyridine-2-yl)methoxy group or a substituted (1,2,3-triazol-4-yl)methoxy group.

Processes which may be used to synthesise the compounds of formula I and, in specifically, incorporate a radiolabel into compounds of the invention, are described above. In particular, process (ii) for the preparation of a compound of formula I is an example of “click” chemistry which may be used to transform a terminal alkyne into a 1,2,3-triazole comprising a pendant radiolabelled functional group. Huisgen cycloadditions such as these and other similar processes are well known to the skilled person under the term “click” chemistry. These are processes which may allow the rapid and reliable formation of target chemical substances from small molecule precursors, processes which are advantageous in the incorporation of radioactive nuclei organic structures.

Analogous reactions to those described in process (ii) for the preparation of compounds of formula I include reactions in which the terminal alkyne of the starting material in that process is replaced with a disubstituted alkyne or a mono- or di-substituted alkene. Such process may also include reactions in which the azide is substituted with another 1,3-dipolar compound, including, but not limited to a diazoalkane, a nitril oxide, ozone or an allene. The skilled person would appreciate that alternative catalysts and reaction conditions may be required for such processes. Examples of such processes may be found in V. V. Rostovtsev, et al., Angew. Chem. Int. Ed., 2002, 41, 2596-2599; D. Amantini, et al., J. Org. Chem. 2005, 70, 6526-6529; J. E. Wilson, et al., Angew. Chem. Int. Ed., 2006, 45, 1426-1429; Z. Liu, et al., J. Org. Chem., 2008, 73, 219-226; and J. Xu, et al., Synlett, 2008, 919-923.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Thus, the compounds of the invention are useful because they possess pharmacological activity, and/or are metabolised in the body following oral or parenteral administration to form compounds which possess pharmacological activity.

According to a further aspect of the present invention, there is provided a method of treatment of a disease which is associated with, and/or which can be modulated by inhibition of epidermal growth factor receptor tyrosine kinase activity and/or a method of treatment of a disease in which inhibition of epidermal growth factor receptor tyrosine kinase activity desired and/or required (e.g. breast cancer), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known types of pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Preferred pharmaceutical formulations include those in which the active ingredient is present in at least 1% (such as at least 10%, preferably in at least 30% and most preferably in at least 50%) by weight. That is, the ratio of active ingredient to the other components (i.e. the addition of adjuvant, diluent and carrier) of the pharmaceutical composition is at least 1:99 (e.g. at least 10:90, preferably at least 30:70 and most preferably at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

According to a further aspect of the invention, there is provided a combination product comprising:
(A) a compound of the invention, as hereinbefore defined; and
(B) another therapeutic agent that is useful in the inhibition of epidermal growth factor receptor tyrosine kinase activity,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the inhibition of epidermal growth factor receptor tyrosine kinase activity, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the inhibition of epidermal growth factor receptor tyrosine kinase activity in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the inhibition of epidermal growth factor receptor tyrosine kinase activity, and at least one pharmaceutically-acceptable adjuvant, diluent or carrier.

By “bringing into association”, we mean that the two components are rendered suitable for administration in conjunction with each other.

According to a further aspect of the invention, there is provided a combination product comprising:

(A) a compound of the invention, as hereinbefore defined; and
(B) an ABC transporter inhibitor,
wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, an ABC transporter inhibitor, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including an ABC transporter inhibitor in admixture with a pharmaceutically-acceptable adjuvant, diluent or carrier,
   which components (a) and (b) are each provided in a form that is suitable for administration in conjunction with the other.

The invention further provides a process for the preparation of a combination product as defined above, which process comprises bringing into association a compound of the invention, as hereinbefore defined, or a pharmaceutically acceptable salt thereof with an ABC transporter inhibitor, and at least one pharmaceutically-acceptable adjuvant, diluent or carrier.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, the nature and location of the tissues or organs to be imaged, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

The invention will now be described in more detail by reference to the following Examples and Figures.

In the Figures:

FIG. 1 shows immunoblots demonstrating inhibition of EGFR autophosphorylation. Cellular activity of quinolines 1 and 17 assessed by Western blots analysis of phosphorylated EGFR (p-EGFR) and total EGFR (EGFR).

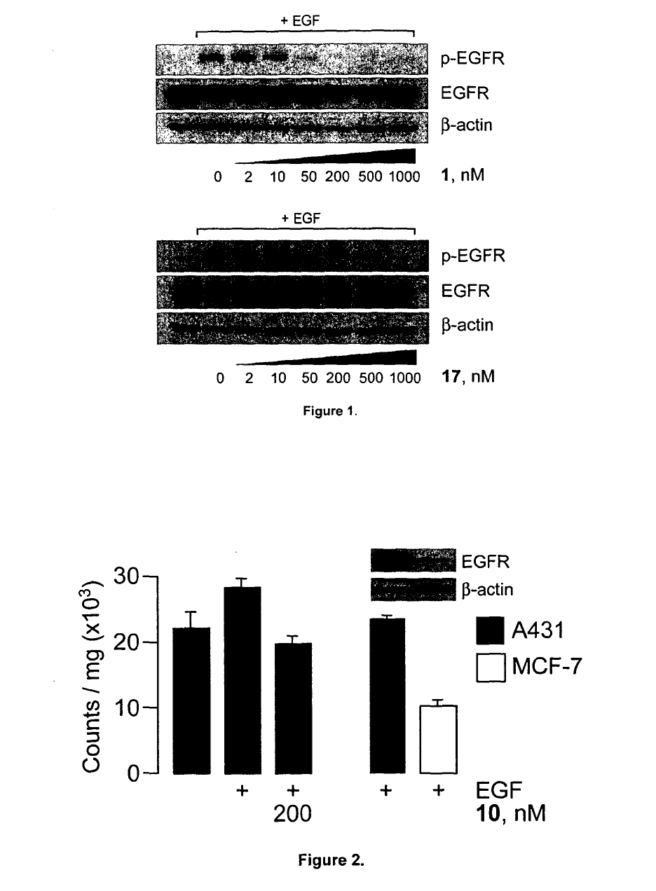

FIG. 2 shows compound [¹⁸F]17 cell uptake in A431 cells. Data were expressed as decay-corrected counts per min per mg total cellular protein. Data are mean±SEM done in triplicate.

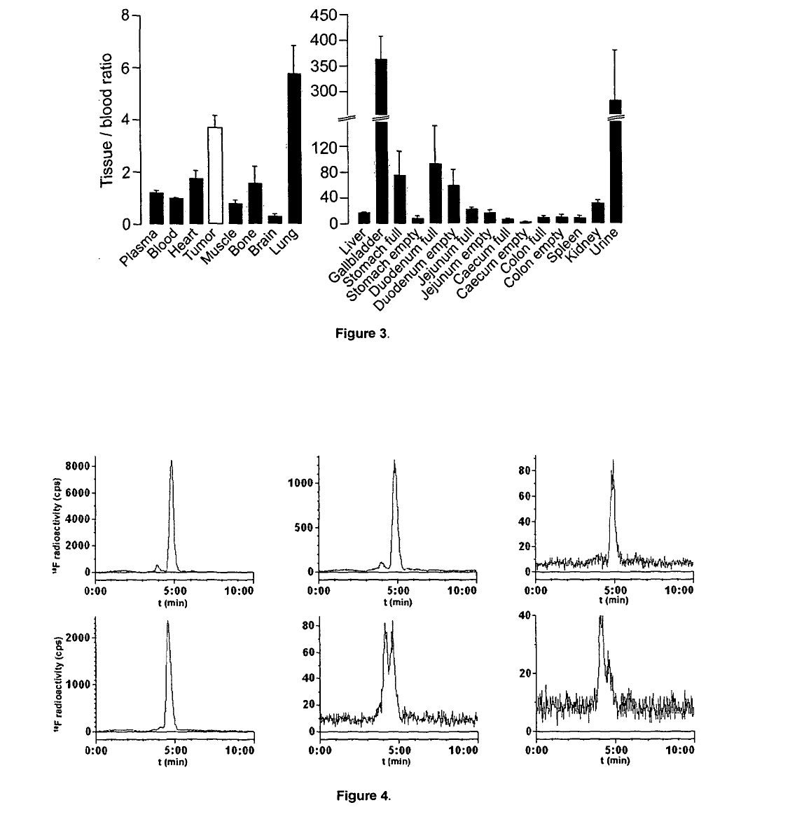

FIG. 3 shows tissue distribution of compound [¹⁸F]17 in untreated tumor bearing mice expressed as tissue to blood ratios at 60 min. Data are ±SEM; n=3 mice.

FIG. 4 shows radiochromatograms obtained as part of the investigation of in vivo metabolic stability of [¹⁸]F17. In vivo metabolism of compound [¹⁸F]17 as assessed by radio-HPLC. Top line: 2 min, 30 min and 60 min liver, respectively; Bottom line: 2 min, 30 min and 60 min plasma, respectively.

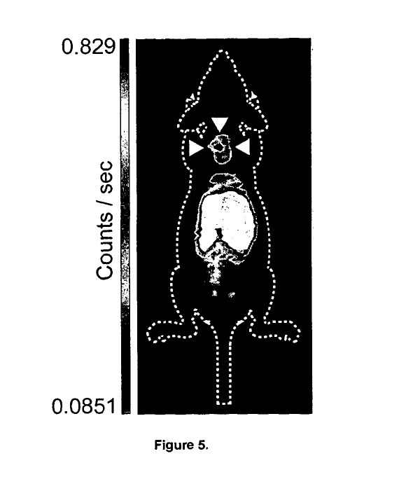

FIG. 5 shows compound [¹⁸F]17 PET image of one representative A431 xenograft-bearing mouse, white arrowheads indicate the tumor.

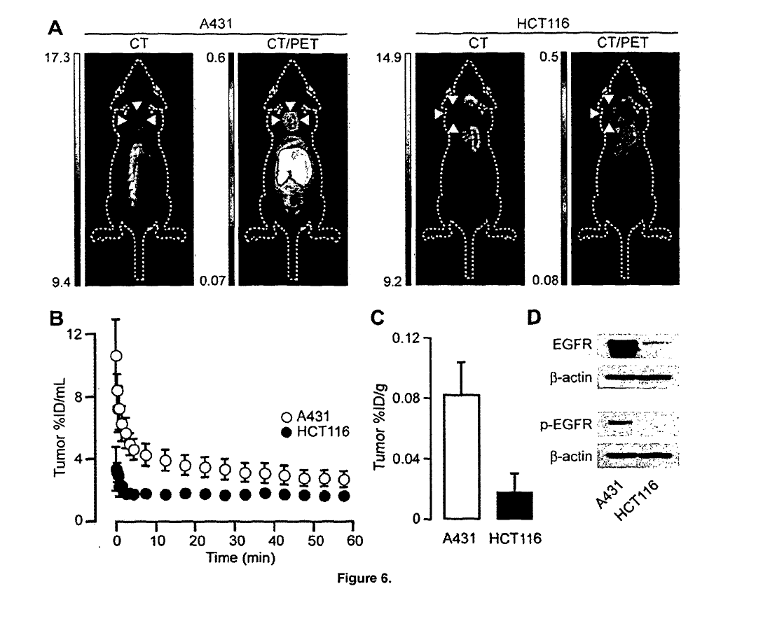

FIG. 6 shows compound [¹⁸F]17 PET images (summed dynamic) of representative A431 and HCT116 xenograft-bearing mice (A), time activity curves (TACs) of A431 and HCT116 tumours (B), tumour uptake measured by γ-counting (C) and western blot of the two cell lines for EGFR and phosphorylated EGFR-p-EGFR; β-actin used as loading control (D).

### EXAMPLES

The invention is illustrated by way of the following examples, in which the following abbreviations may be employed:

DMF dimethylformamide
MeOH methanol
MeCN acetonitrile
THF tetrahydrofuran
DMSO dimethylsulfoxide
NMR nuclear magnetic resonance
MS Mass spectrometry

### ESI Electrospray

IR Infrared spectroscopy
TLC thin layer chromatography
HPLC high performance liquid chromatography
rt room temperature
PET positron emission tomography
EGFR epidermal growth factor receptor
PTFE polytetrafluoroethylene
PBS phosphate-buffered saline
ATP adenosine triphosphate
EDTA ethylenediaminetetraacetic acid

### DMEM Dulbecco's Modified Eagle's Medium

CT computed tomography
Boc tert-butyloxycarbonyl

### Example 1

The quinoline advanced intermediate 6, the sugar derivative 27, and the Michael acceptors 3, 4 and 5 and quinoline based EGFR inhibitor 1 were synthesized accordingly to literature procedures: Wissner A., et al., J. Med. Chem. 2003, 46, 49-63; Kovác, P. Carbohyd. Res. 1986, 153, 168-170; Maschauer, S.; Prante, O. Carbohyd. Res. 2009, 344, 753-761; Tsou H.-R., et al., J. Med. Chem. 2005, 48, 1107-1131; and Wei X., et al., Tetrahedron Lett. 1998, 39, 3815-3818.

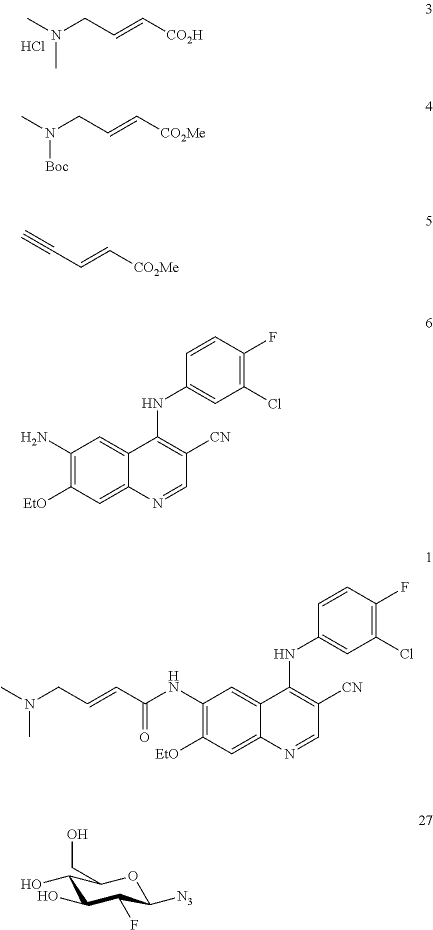

### Literature Compounds Prepared for this Work

### Example 2

Michael acceptor 8 was obtained by reacting commercially available methyl 4-bromocrotonate (7) with propargyl amine at −20° C. then protecting in situ the resulting secondary amine as a Boc carbamate (Scheme 1).

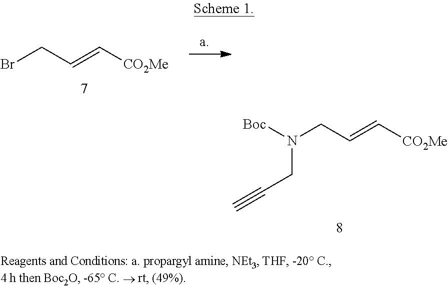 [SMILES: COC(=O)/C=C/CBr ; C#CCN(C/C=C/C(=O)OC)C(=O)OC(C)(C)C]

(E)-4-(tert-Butoxycarbonyl-prop-2-ynylamino)-but-2-enoic acid methyl ester (8): 4-Bromo methylcrotonate (7, 1 g, 5.6 mmol) was dissolved in dry THF (10 mL) and propargyl amine (961 μL, 14 mmol) was added dropwise at −20° C. The resulting mixture was stirred at −20° C. for 4 h then cooled to −65° C. Boc₂O (4.9 g, 22.3 mmol) and Et₃N (4 mL, 27.9 mmol) were then added in turn and the mixture stirred at −65° C.→rt for 14 h. The white solid was filtered off and the mother liqueur was concentrated under reduced pressure, dissolved in CH₂Cl₂ (30 mL) and washed with water (20 mL), HCl 1M (20 mL), water (20 mL) and brine (20 mL) and finally dried over MgSO₄. The crude residue was purified by chromatography on silica gel (Et₂O/petroleum ether, 1:4; R_{f}=0.12) to give the title compound (681 mg, 49%) as colourless oil.

¹H NMR (400 MHz, CDCl₃) δ 6.90 (dt, J=15.7, 5.3, 1H), 5.93 (d, J=15.7, 1H), 4.19-3.91 (m, 4H), 3.77 (s, 3H), 2.24 (t, J=2.4, 1H), 1.49 (s, 9H); ¹³C NMR (101 MHz, CDCl₃) δ 166.5 (s), 154.6 (s), 143.6 (d), 122.1 (d), 121.8 (d), 81.0 (s), 78.9 (d), 72.3 (s), 71.9 (s), 51.7 (q), 47.0 (t), 46.8 (t), 36.4 and 35.9 (t), 28.3 (q, 3C); IR: νₘₐₓ 3263, 2976, 2361, 1699, 1450, 1273, 1167 cm⁻¹; MS (ESI): m/z (%) 276 [MNa⁺] (35); HR-MS (ESI) Calcd for C₁₃H₁₉NO₄Na: 276.1211, found 276.1212 (Δ−0.4 ppm).

### Example 3

The coupling at C-6 of quinoline 6 and methyl esters 4, 5 and 8 was performed by AlMe₃ mediated amidation using dry CH₂Cl₂ to give amides 9, 11 and 12 in yields of 47%, 68% and 20%, respectively. The Boc group in compound 10 was removed with 10% conc. HCl in dioxane and the product precipitated as the hydrochloride salt. This salt was neutralized by treating with K₂CO₃ in H₂O overnight, during which time the free amine 10 precipitated from the solution in 78% yield (Scheme 2). Quinoline 10 was obtained spectroscopically pure and used in the following step with the need of further purifications.

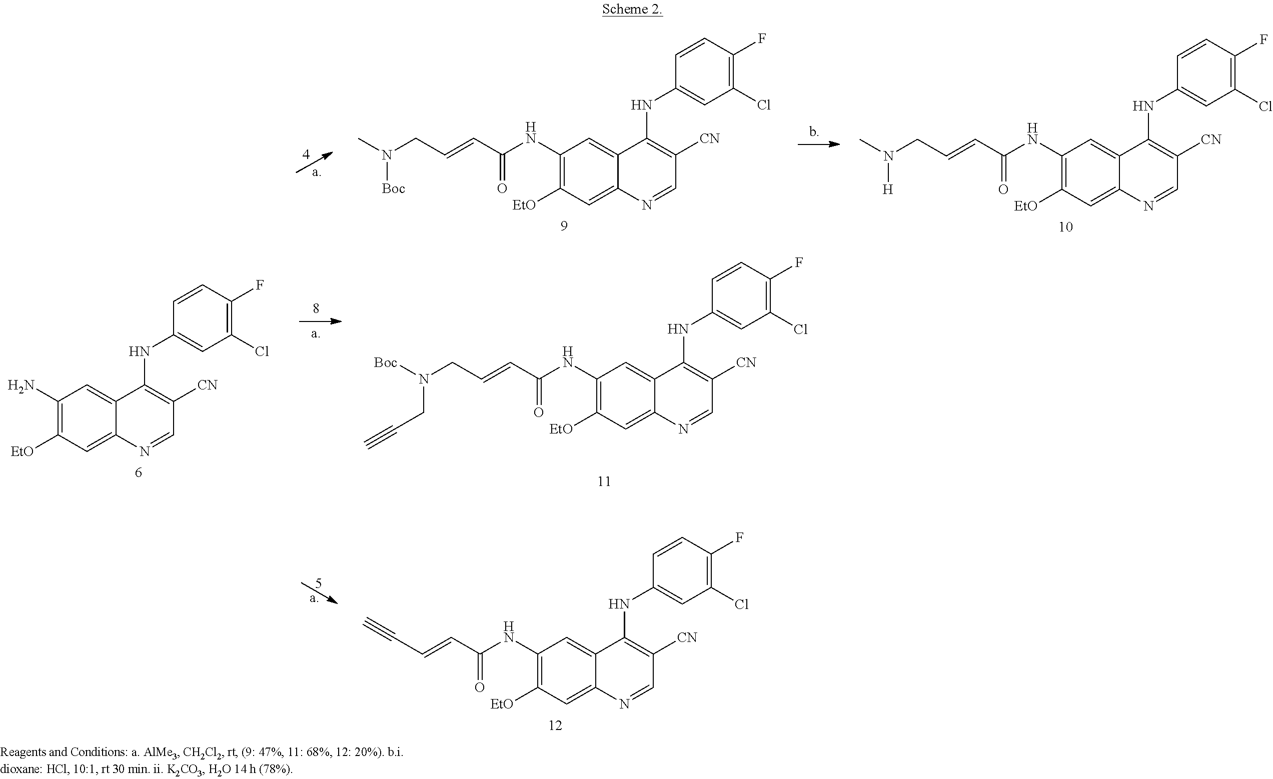 [SMILES: CCOc1cc2ncc(C#N)c(Nc3ccc(F)c(Cl)c3)c2cc1NC(=O)/C=C/CN(C)C(=O)OC(C)(C)C ; CCOc1cc2ncc(C#N)c(Nc3ccc(F)c(Cl)c3)c2cc1NC(=O)/C=C/CNC ; CCOc1cc2ncc(C#N)c(Nc3ccc(F)c(Cl)c3)c2cc1N ; C#CCN(C/C=C/C(=O)Nc1cc2c(Nc3ccc(F)c(Cl)c3)c(C#N)cnc2cc1OCC)C(=O)OC(C)(C)C ; C#C/C=C/C(=O)Nc1cc2c(Nc3ccc(F)c(Cl)c3)c(C#N)cnc2cc1OCC]

{(E)-3-[4-(3-Chloro-4-fluorophenylamino)-3-cyano-7-ethoxyquinolin-6-ylcarbamoyl]-allyl}-methylcarbamic acid isopropyl ester (9): yellow semisolid, 47% yield; R_{f}=0.12 (petroleum ether/AcOEt: 1,2); ¹H NMR (400 MHz, CDCl₃): δ 9.20 (s, 1H), 8.53 (s, 1H), 8.07 (s, 1H), 7.91 (br s, 1H), 7.31-7.21 (m, 1H), 7.21-7.13 (m, 1H), 7.12-6.88 (m, 3H), 6.15-6.02 (m, 1H), 4.38-4.27 (m, 2H), 4.08 (br s, 2H), 2.98-2.85 (m, 3H), 1.61 (t, J=6.9 Hz, 3H), 1.53 (s, 9H); ¹³C NMR (101 MHz, CDCl₃): δ 164.4 (s), 156.6 [s (d, J_{CF}=247.8 Hz], 155.6 (s), 152.0 (d), 150.8 (s), 149.4 (s), 147.2 (s), 143.3 and 142.3 (d), 135.7 (s), 127.3 (s), 125.8 (d), 124.3 (d), 123.1 (d), 121.1 [s, (d, J_{CF}=18.9 Hz), 116.8 (s), 116.4 [d (d, J_{CF}=22.3 Hz)], 113.1 (s), 109.9 and 109.5 (d), 108.4 (d), 88.3 (s), 79.9 (s), 65.0 (t), 49.9 and 49.3 (t), 34.3 (q), 28.2 (q, 3C), 14.4 (q); MS (ESI): m/z (%) 554 [MH⁺] (100); HR-MS (ESI) Calcd for C₂₈H₃₀ClFN₅O₄: 554.1970, found 554.1981 (2.0 ppm).

(E)-4-(Methylamino)-but-2-enoic acid [4-(3-chloro-4-fluorophenylamino)-3-cyano-7-ethoxyquinoline-6-yl] amide hydrochloride (10.HCl): yellow solid; 78% yield; ¹H NMR (400 MHz, MeOD): δ 9.24 (s, 1H), 8.87 (s, 1H), 7.72 (dd, J=6.5, 2.5 Hz, 1H), 7.52 (ddd, J=6.5, 8.7, 2.5, 1H), 7.48-7.36 (m, 2H), 7.00 (dt, J=15.2, 1H), 6.82 (dt, J=15.2, 1.4, 1H), 4.47 (q, J=7.0, 2H), 3.92 (d, J=6.4, 2H), 2.78 (s, 3H), 1.62 (t, J=7.0, 3H); ¹³C NMR (101 MHz, MeOD): δ 163.5 (s), 158.1 [s (d, J_{CF}=250.1 Hz], 155.7 (s), 154.5 (s), 147.5 (d), 146.1 (s), 136.6 (s), 134.6 (d), 133.8 (s), 129.6 (d), 129.4 (d), 127.7 [d (d, J_{CF}⁼7.8 Hz)], 121.3 [s (d, J_{CF}=19.2)], 117.0 [d (d, J_{CF}=22.7 Hz)], 114.0 (d), 113.1 (s), 111.9 (s), 100.3 (d), 86.7 (s), 66.8 (t), 48.9 (t), 32.2 (q), 13.2 (q); MS (ESI): m/z (%) 454 [MH⁺] (48), 248 (100); HR-MS (ESI) Calcd for C₂₃H₂₂ClFN₅O₂: 454.1446, found 454.1459 (2.9 ppm).

(E)-4-(Methylamino)-but-2-enoic acid [4-(3-chloro-4-fluorophenylamino)-3-cyano-7-ethoxy-quinoline-6-yl]amide (10): The quinoline hydrochloride 10.HCl (39.0 mg, 0.08 mmol) was dissolved in water (1 mL) and K₂CO₃ (55 mg, 0.4 mmol) was added. The mixture was stirred 14 h at rt and the pale yellow precipitate was collected, washed with water and dried under vacuum to give the title compound 10 (27.1 mg, 78%) as a yellow solid.

¹H NMR (400 MHz, MeOD): δ 8.96 (s, 1H), 8.48 (s, 1H), 7.48-7.41 (m, 1H), 7.41-7.35 (m, 1H), 7.35-7.24 (m, 2H), 7.04 (dt, J=15.4, 5.8 Hz, 1H), 6.50 (dt, J=15.5, 1.5 Hz, 1H), 4.37 (q, J=6.8 Hz, 2H), 3.44 (dd, J=5.7, 1.0 Hz, 2H), 2.45 (s, 3H), 1.59 (t, J=6.9 Hz, 3H).

{(E)-3-[4-(3-Chloro-4-fluoro-phenylamino)-3-cyano-7-ethoxy-quinolin-6-ylcarbamoyl]-allyl}-prop-2-ynyl-carbamic acid tert-butyl ester (11): colourless oil; 68% yield, R_{f}=0.14 (eluent: AcOEt/Et₂O, 10/1); ¹H NMR (400 MHz, CDCl₃) δ 9.18 (s, 1H), 8.39 (s, 1H), 8.17 (br s, 1H), 7.96 (s, 1H), 7.08 (s, 1H), 7.05-6.94 (m, 2H), 6.89 (t, J=8.6, 1H), 6.68 (m, 1H), 6.08 (d, J=14.9, 1H), 4.22 (q, J=6.9, 2H), 4.13 (d, J=4.2, 2H), 4.05 and 3.90 (br s, 2H), 2.25 (t, J=2.2, 1H), 1.58 (t, J=7.0, 3H), 1.48 (s, 9H); ¹³C NMR (101 MHz, CDCl₃) δ 164.3 (s), 155.8 [s (d, J_{CF}=249.8 Hz], 154.7 (s), 152.1 (d), 151.0 (s), 149.6 (s), 147.3 (s), 142.7 and 142.2 (d), 135.7 (s), 127.5 (s), 126.1 (d), 125.5 (d), 123.43 and 123.36 (d), 121.2 [s; (d, J=18.9)], 116.8 (s), 116.5 [d, (d, J=22.3)]; 113.2 (s), 109.7 (d), 108.5 (d), 88.5 (d), 81.0 (s), 79.1 (s), 72.2 and 71.9 (d), 65.1 (t), 47.0 (t), 36.5 and 35.9 (t), 28.3 (q; 3C), 14.1 (q); MS (ESI): m/z (%) 578 [MW] (100); IR: νₘₐₓ 3412, 3307, 2980, 2930, 2252, 2214, 1690, 1682, 1537, 1250, 734 cm⁻¹; HR-MS (ESI) Calcd for C₃₀H₃₀N₅O₄FCl: 578.1970, found 578.1948 (Δ−3.8 ppm).

(E)-Pent-2-en-4-ynoic acid [4-(3-chloro-4-fluorophenylamino)-3-cyano-7-ethoxyquinolin-6-yl]-amide (12): yellow solid, 20% yield, R_{f}=0.13 (Et₂O); ¹H NMR (500 MHz, CDCl₃) δ 9.10 (s, 1H), 8.56 (s, 1H), 8.09 (s, 1H), 7.39 (s, 1H), 7.35 (br s, 1H), 7.24 (dd, J=6.3, 2.7, 1H), 7.16 (t, J=8.6, 1H), 7.08 (ddd, J=2.8, 3.9, 8.7, 1H), 6.82 (dd, J=1.6, 15.4, 1H), 6.55 (d, J=15.4, 1H), 4.33 (q, J=7.0, 2H), 3.40 (dd, J=0.4, 2.4, 1H), 1.58 (t, J=7.0, 3H); ¹³C NMR (126 MHz, CDCl₃) δ 162.6 (s), 156.6 [s, (d, J_{CF}=249.0), 152.2 (d), 151.3 (s), 150.0 (s), 147.6 (s), 135.6 (s), 134.2 (d), 128.1 (s), 126.9 (d), 124.6 [d, (d, J_{CF}=7.1)], 122.9 (d), 121.9 [s, (d, J_{CF}=19.1)], 117.2 [d, (d, J_{CF}=22.3)], 116.5 (s), 113.2 (s), 109.9 (d), 108.8 (d), 89.2 (s), 86.3 (d), 80.3 (s), 65.3 (t), 14.5 (q); ¹⁹F NMR (376 MHz, CDCl3) δ−117.0 ppm; IR: νₘₐₓ 3300, 2925, 2361, 2342, 2214, 1670, 1624, 1539, 1498, 1458, cm⁻¹; MS (ESI): m/z (%) 435 [MW] (100); HR-MS (ESI) Calcd for C₂₃H₁₇N₄O₂FCl: 435.1024, found 435.1018 (Δ−1.4 ppm).

{(E)-3-[4-(3-Chloro-4-fluoro-phenylamino)-3-cyano-7-(2-fluoroethoxy)-quinolin-6-ylcarbamoyl]-allyl}-methylcarbamic acid tert-butyl ester (Boc-24): yellow oil, 61% yield; R_{f} 0.38 (AcOEt); ¹H NMR (500 MHz, CDCl₃) δ 9.15 (s, 1H), 8.57 (s, 1H), 8.10 (s, 1H), 7.40 (s, 1H), 7.31-7.25 (m, 1H), 7.21-7.14 (m, 1H), 7.14-7.07 (m, 1H), 6.93 (dt, J=5.0, 15.2, 1H), 6.13-5.98 (m, 1H), 4.90 (dm, J_{HF}=47.9, 2H), 4.50 (dm, J_{HF}=27.3, 2H), 4.06 (br s, 2H), 2.91 (br s, 3H), 1.48 (s, 9H); ¹³C NMR (126 MHz, CDCl₃) δ 163.6 (s), 156.6 [s, (d, J_{CF}=284.1)], 155.8 (s), 152.3 (d), 150.6 (s), 150.0 (s), 147.1 (s), 142.6 and 142.2 (d), 135.5 (s), 128.4 (s), 126.9 (d), 124.6 (d), 123.5 (d), 121.8 [s, (d, J_{CF}=117.2 [d, (d, J_{CF}=22.3)], 116.5 (s), 113.8 (s), 110.0 (d), 109.2 (d), 100.0 (s), 85.4 [t (d, J_{CF}=174.1)], 80.9 (s), 68.4 [t (d, J_{CF}=19.5)], 50.0 and 49.4 (t), 34.6 and 34.5 (q), 29.7 (s, 3C); MS (ESI): m/z (%); 572 [MW] (100); HR-MS (ESI) Calcd for C₂₈H₂₉N₅O₄ClF₂: 572.1876, found 572.1868 (Δ−1.4 ppm).

### Example 4

Derivatisation of N-methyl amine 10 was achieved by two methods: alkylation and reductive amination. Alkylation of amine 10 with 1-mesyloxy-2-fluoro ethane (15) in CH₂Cl₂ gave the N-fluoroethyl product 13 in 33% yield along with unidentified by-products which made the final purification difficult and limited the yield. As the N-alkylation reaction could not be developed into an efficient method to introduce the desired fluorine-contained substituent, reductive amination was explored as an alternative method. Consequently, quinoline 10 was transformed into 4-fluorobenzyl product 14 by treatment with 4-fluoro benzaldehyde and NaBH(OAc)₃. Compound 14 was obtained in a 21% unoptimized yield (Scheme 3).

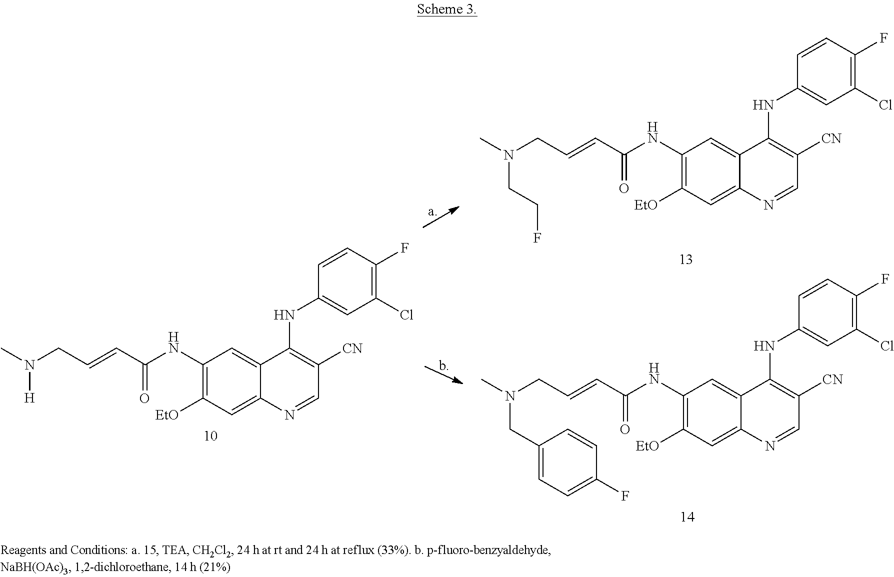 [SMILES: CCOc1cc2ncc(C#N)c(Nc3ccc(F)c(Cl)c3)c2cc1NC(=O)/C=C/CN(C)CCF ; CCOc1cc2ncc(C#N)c(Nc3ccc(F)c(Cl)c3)c2cc1NC(=O)/C=C/CNC ; CCOc1cc2ncc(C#N)c(Nc3ccc(F)c(Cl)c3)c2cc1NC(=O)/C=C/CN(C)Cc1ccc(F)cc1]

(E)-4-[(2-Fluoroethyl)methyl amino]-but-2-enoic acid [4-(3-chloro-4-fluorophenylamino)-3-cyano-7-ethoxyquinoline-6-yl]amide (13): Quinoline 10 (32 mg, 0.07 mmol) was dissolved in dry CH₂Cl₂ (0.7 mL) and 1-mesyloxy-2-fluoro ethane (16, 13 mg, 0.09 mmol) and triethylamine (20 μL, 0.14 mmol) were added in turn. The mixture was stirred overnight, concentrated and directly purified by preparative silica TLC on silica gel (CH₂Cl₂/MeOH, 20:1; R_{f}=0.28) to give quinoline 13 (11.5 mg, 33%) as a yellow solid.

¹H NMR (400 MHz, CDCl₃): δ 9.17 (s, 1H), 8.54 (s, 1H), 8.11 (s, 1H), 7.53 (s, 1H), 7.33 (s, 1H), 7.21 (dd, J=6.3, 2.6, 1H), 7.12 (t, J=8.6, 1H), 7.08-6.99 (m, 2H), 6.27 (dt, J=15.2, 1.6, 1H), 4.59 (dt, J=47.6, 4.8, 1H), 4.32 (q, J=7.0, 2H), 3.33 (dd, J=5.6, 3.1, 2H), 2.77 (dt, J=28.0, 4.8, 2H), 2.39 (s, 3H), 1.60 (t, J=7.0, 3H); ¹³C NMR (126 MHz, CDCl₃): δ 164.0 (s), 156.4 [s (d, J_{CF}⁼248.7 Hz)], 152.2 (d), 151.2 (s), 149.9 (s), 147.4 (s), 143.9 (d), 135.7 (s), 128.3 (s), 126.7 (d), 125.3 (d), 124.3 [d (d, J_{CF}=7.3 Hz)], 121.7 [s, J_{CF}=19.0)], 117.1 (s), 116.8 [d (d, J_{CF}=24.8 Hz)], 113.3 (s), 109.4 (d), 108.8 (d), 88.9 (s), 82.1 [t, (d, J_{CF}=168.0 Hz)], 65.2 (t), 58.6 (t), 57.0 [t, (d, J_{CF}=19.7 Hz)], 42.9 (q), 14.5 (q); ¹⁹F NMR (376 MHz, CDCl₃): δ−117.5, −219.4; MS (ESI): m/z (%) 500 [MH⁺] (76), 271 (100); IR: νₘₐₓ 3250, 2924, 2212, 1685, 1622, 1537, 1498, 1458, 1393, 1215 cm⁻¹; HR-MS (ESI) Calcd for C₂₅H₂₅N₅O₂F₂Cl: 500.1665, found 500.1662 (Δ−0.6 ppm).

(E)-4-[(4-Fluorobenzyl)methylamino]-but-2-enoic acid [4-(3-chloro-4-fluorophenylamino)-3-cyano-7-ethoxyquinoline-6-yl]amide (14): Quinoline 10 (50 mg, 0.10 mmol) was suspended in dry dichloroethane (0.3 mL) and p-fluorobenzaldehyde (12 μL, 0.11 mmol) and acetic acid (10 μL) was added dropwise at rt. After 30 min, NaBH(OAc)₃ (32 mg, 0.15 mmol) was added and the mixture was stirred 14 hat rt. The mixture was quenched with a saturated solution of NaHCO₃ (1 mL) and extracted with CH₂Cl₂ (3×2 mL). The combined organic layers were dried over MgSO₄. After purification by plate silica TLC (CH₂Cl₂/MeOH, 20:1; R_{f}=0.37), the title compound 14 was obtained (12 mg, 21%) as a yellow oil.

¹NMR (400 MHz, CDCl₃): δ 9.18 (s, 1H), 8.52 (s, 1H), 8.07 (s, 1H), 7.66 (s, 1H), 7.34-7.27 (m, 4H), 7.12 (dd, J=6.3, 2.5, 1H), 7.12-7.00 (m, 4H), 6.96 (dt, J=8.3, 3.4, 1H), 6.24 (dt, J=15.3, 1.6, 1H), 4.32 (q, J=7.0, 2H), 3.53 (s, 2H), 3.23 (dd, J=5.7, 1.0, 2H), 2.25 (s, 3H), 1.61 (t, J=6.7, 3H); ¹³C NMR (126 MHz, CDCl₃): δ 163.9 (s), 162.1 [s, J_{CF}⁼245.1 Hz], 156.5 [s (d, J_{CF}=249.2 Hz], 152.1 (d), 151.2 (s), 149.8 (s), 147.5 (s), 144.5 (d), 135.7 (s), 134.2 (s), 130.3 [d, (d, J_{CF}=7.8 Hz, 20)], 128.5 (s), 126.8 (d), 125.2 (d), 124.3 [d (d, J_{CF}=7.4 Hz)], 121.6 [s, (d, J_{CF}=21.0)], 117.2 (s), 116.9 [d (d, J_{CF}=43.3 Hz)], 115.2 [d (d, J_{CF}=21.4 Hz, 20)], 113.3 (s), 109.3 (d), 108.8 (d), 89.1 (s), 65.2 (t), 61.3 (t), 57.8 (t), 42.5 (q), 14.6 (q); ¹⁹F NMR (376 MHz, CDCl₃): δ−117.6, −115.5; MS (ESI): m/z (%) 562 [MH⁺] (50), 454 (100); IR: νₘₐₓ 3380, 2922, 2220, 1680, 1620, 1537, 1458 cm⁻¹; HR-MS (ESI) Calcd for C₃₀H₂₇N₅O₂F₂Cl: 562.1821, found 562.1828 (1.2 ppm).

### Example 5

Quinoline precursor 11 was reacted with 1-fluoro-2-ethyl azide (16) under Cu(I) catalysis and microwave irradiation to give the Boc protected analogue of quinoline 17 (Boc-17) which was treated with HCl in 1,4-dioxane to form the final quinoline 17 as an HCl salt. (Scheme 4a). The preparation and isolation of Boc-17 is also described in Example 6.

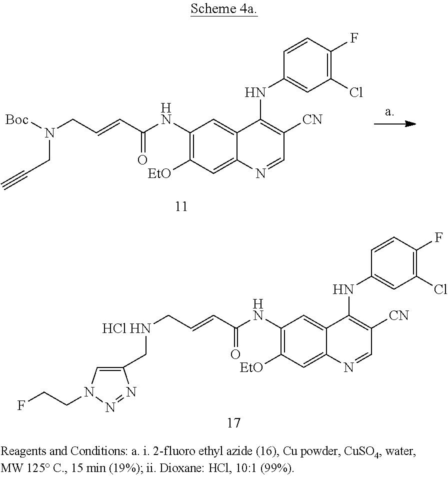

(E)-4-{[1-(2-Fluoro-ethyl)-1H-[1,2,3]triazol-4-ylmethyl]-amino}-but-2-enoic acid [4-(3-chloro-4-fluoro-phenylamino)-3-cyano-7-ethoxy-quinolin-6-yl]-amide hydrochloride (17): yellow solid; 99%; ¹H NMR (500 MHz, d₆-DMSO) δ 10.99 (br s, 1H), 9.93 (s, 1H), 9.83 (s, 1H), 9.12 (br s, 1H), 8.97 (s, 1H), 8.33 (s, 1H), 7.72 (d, J=6.1, 1H), 7.61 (s, 1H), 7.53 (t, J=9.0, 1H), 7.47-7.41 (m, 1H), 6.87 (dt, J=15.5, 6.35, 1H), 6.78 (d, J=15.6, 1H), 4.84 (dm, J=32.3, 2H), 4.79-4.74 (m, 2H), 4.34 (q, J=7.0, 2H), 4.29 (t, J=4.9, 2H), 3.86 (br dd, J=11.6, 5.9, 2H), 1.49 (t, J=7.0, 3H); ¹⁹F NMR (376 MHz, d₆-DMSO) δ−117.9, −222.0; ¹³C NMR (101 MHz, d₆-DMSO) δ 162.7 (s), 155.3 [s (d, J_{CF}=422.7)] 155.1 (s), 154.9 (s), 149.3 (d), 138.5 (s), 135.6 (s), 134.6 (d), 129.4 (d), 128.6 (s), 128.4 (s), 127.8 [d (d, J_{CF}=7.5)], 126.6 (d), 126.0 (d), 119.8 Es, (d, J_{CF}=18.9)], 117.3 [d, (d, J_{CF}=22.3)], 116.4 (d), 114.8 (s), 112.5 (s), 102.9 (d), 86.9 (s), 81.9 [t, (d, J_{CF}=168.3)], 65.3 (t), 50.2 [t, (d, J_{CF}=19.4)], 46.6 (t), 40.8 (t), 14.1 (q); MS (ESI): m/z (%) 567 [MH⁺] (80), 440 (100); HR-MS (ESI) Calcd for C₂₇H₂₆N₈O₂F₂Cl: 567.1835, found 567.1841 (Δ1.1 ppm).

Quinoline precursor 11 was reacted with 1-azido-2-deoxy-2-fluoro-D-glucose (27) under Cu(I) catalyzed Huisgen 1,3-dipolar cycloaddition (‘Click’ chemistry) to give the Boc protected analogue of quinoline 28 (Boc-28) (Scheme 4b).

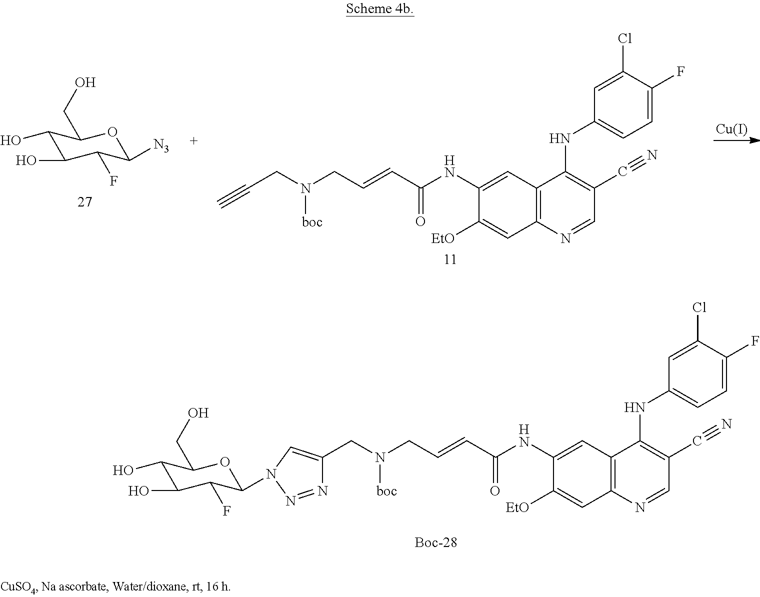

{(E)-3-[4-(3-Chloro-4-fluoro-phenylamino)-3-cyano-7-ethoxy-quinolin-6-ylcarbamoyl]-allyl}-(1-(3-fluoro-4,5-dihydroxy-6-hydroxymethyl-tetrahydro-pyran-2-yl)-1H-[1,2,3]triazol-4-ylmethyl]-carbamic acid tert-butyl ester (Boc-28): yellow semisolid, 70% yield, R_{f}=0.34 (eluent: AcOEt/MeOH, 10:1); ¹H NMR (400 MHz, CDCl₃) δ 8.94 (s, 1H), 8.54 (br s, 1H), 8.20 (s, 1H), 7.40 (dd, J=6.5, 1.9, 1H), 7.37-7.30 (m, 1H), 7.28-7.19 (m, 2H), 6.93-6.78 (m, 1H), 6.48-6.33 (m, 1H), 5.92 (d, J=9.3, 1H), 4.81 (dt, J_{HF}=48.9, J_{HH}=9.1, 1H), 4.65-4.53 (m, 2H), 4.33 (q, J=7.0, 2H), 4.21-4.06 (m, 2H), 3.89-3.77 (m, 2H), 3.71-3.59 (m, 2H), 3.58-3.51 (m, 1H), 1.56 (t, J=6.8, 3H), 1.47 (s, 9H); ¹³C NMR (101 MHz, CDCl₃) δ 173.0 (s), 166.0 (s), 158.8 (s), 156.4 (s), 154.8 (s), 154.7 [s, (d, J_{CF}=432.2)], 153.5 (s), 142.6 (d), 138.1 (s), 129.5 (s), 128.0 (d), 126.2 [d, (d, J_{CF}=7.6)], 125.9, 125.5 (d), 124.3, 123.8 (d), 122.23 [s, (d, J_{CF}=19.3)], 118.1 [d, (d, J_{CF}=22.6)], 117.8 (s), 114.6 (d), 108.8 (s), 92.1 [d, (d, J_{CF}=188.0)], 86.5 [d, (d, J_{CF}=24.5)], 82.2 (s), 81.2 (d), 76.4 [d, (d, J_{CF}=16.7)], 70.1 [d, (d, J_{CF}=7.7)], 66.3 (t), 62.2 (t), 49.8 (t), 43.4, 42.8 (t), 28.6 (q, 3C), 14.7 (q); HR-MS (ESI) Calcd for C₃₆H₄₀ClF₂N₈O₈: 785.2626, found 785.2641 (Δ1.9 ppm); MS (ESI): m/z (%) 785 [MH⁺] (100);

### Example 6

Derivatisation of the enzyne containing quinoline 12 was effected by “click” cycloaddition with azide 16. This reaction gave quinoline 18 directly without the requirement for any further synthetic manipulations (Scheme 5)

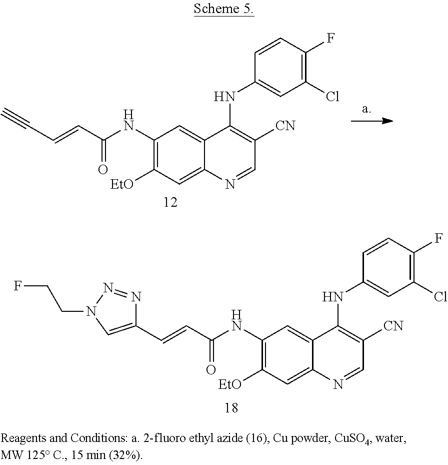 [SMILES: C#C/C=C/C(=O)Nc1cc2c(Nc3ccc(F)c(Cl)c3)c(C#N)cnc2cc1OCC ; CCOc1cc2ncc(C#N)c(Nc3ccc(F)c(Cl)c3)c2cc1NC(=O)/C=C/c1cn(CCF)nn1]

General procedure for the synthesis of compounds Boc-17 and 18: Quinoline 11 or 12 (1 eq) was dispersed in water (0.15 M) and fluoro ethyl azide 15 (0.5 M solution in DMF, 2 eq), CuSO₄ (0.3 eq) and Cu powder (0.3 eq) were added. The mixture was heated by microwave irradiation at 125° C. for 15 min and diluted with water and AcOEt. The phases were separated and the aqueous phase was extracted with AcOEt. The combined organic layers were dried over MgSO₄. The crude residue was purified by chromatography on silica gel to give the compounds Boc-17 or 18 respectively.

{(E)-3-[4-(3-Chloro-4-fluorophenylamino)-3-cyano-7-ethoxyquinolin-6-ylcarbamoyl]-allyl}-(1-(2-fluoroethyl)-1H-[1,2,3]triazol-4-ylmethyl]-carbamic acid tert-butyl ester (Boc-17): yellow semisolid; 19% yield; R_{f}=0.36 (eluent: AcOEt/MeOH, 10:1); ¹H NMR (400 MHz, CDCl₃) δ 9.15 (s, 1H), 8.58 (s, 1H), 8.14 (s, 1H), 7.73 (dd, J=5.7, 3.3, 1H), 7.53-7.42 (m, 2H), 7.31 (dd, J=6.2, 2.6, 1H), 7.21 (t, J=8.5, 1H), 7.17-7.11 (m, 1H), 6.97-6.87 (m, 1H), 6.26-6.06 (m, 1H), 4.81 (dt, J=46.8, 4.3, 2H), 4.69 (dm, J=26.7, 2H), 4.58-4.50 (m, 2H), 4.42-4.34 (m, 2H), 4.22-4.13 (m, 2H), 1.63 (t, J=6.8, 3H), 1.28 (s, 9H); ¹⁹F NMR (376 MHz, CDCl₃) δ−59.9, −115.8; ¹³C NMR (126 MHz, CDCl₃) δ 171.1 (s), 163.7 (s), 156.3 [s (d, J_{CF}=249.0)], 152.1 (d), 151.3 (s), 149.9 (s), 147.3 (s), 145.2 and 144.9 (s), 142.2 and 141.8 (d), 135.6 (s), 128.2 (s), 126.7 (d), 124.4 (d), 124.3 [d (d, J_{CF}=6.8)], 123.8 and 123.0 (d), 121.6 [s (d, J_{CF}=19.1)], 117.0 [d (d, J_{CF}=22.4)], 116.6 (s), 113.2 (s), 109.7 (d), 108.6 (d), 88.9 (s), 81.4 [t (d, J_{CF}=162.1)], 80.8 (s), 65.2 (t), 50.5 [t (d, J_{CF}=20.4)], 47.9 and 47.5 (t), 42.0 and 41.5 (t), 28.3 (q, 3C), 14.5 (q); IR: νₘₐₓ 2925, 2854, 2220, 1688, 1537, 1459, 1163 cm⁻¹; MS (ESI): m/z (%) 689 [MNa⁺] (100), 667 [M⁺]; HR-MS (ESI) Calcd for C₃₂H₃₄N₈O₄F₂Cl: 667.2360, found 667.2354 (Δ−0.9 ppm).

(E)-N-[4-(3-Chloro-4-fluorophenylamino)-3-cyano-7-ethoxyquinolin-6-yl]-3-(1-(2-fluoro-ethyl)-1H-[1,2,3]triazol-4-yl]-acrylamide (18): yellow solid; 32% yield; R_{f}=0.28 (AcOEt/MeOH, 1:1); ¹H NMR (500 MHz, CDCl₃) δ 9.20 (s, 1H), 8.55 (s, 1H), 8.26 (s, 1H), 7.83 (s, 1H), 7.68 (d, J=15.3, 1H), 7.48 (s, 1H), 7.29 (dd, J=6.2, 2.6, 1H), 7.18 (t, J=8.6, 1H), 7.1-7.09 (m, 1H), 7.05 (d, J=15.3, 1H), 4.84 (dm, J=46.7, 2H), 4.73 (dm, J=27.1, 2H), 4.35 (q, J=7.0, 2H), 1.61 (t, J=7.0, 3H); ¹⁹F NMR (376 MHz, CDCl₃) δ−116.1, −220.7; ¹³C NMR (126 MHz, CDCl₃) δ 163.89 (s), 156.73 [s, (d J_{CF}=250.1), 151.62 (d), 151.38 (s), 150.29 (s), 148.88 (s), 143.57 (s), 135.41 (s), 130.30 (d), 128.75 (s), 127.08 (d), 124.92 (d), 124.72 [d (d, J_{CF}=6.4)], 122.24 (d), 121.91 [s (d, J_{CF}=18.9)], 117.23 [d (d, J_{CF}=22.4)], 116.29 (s), 113.17 (s), 109.54 (d), 108.05 (d), 99.96 (s), 81.36 [t (d, J_{CF}=173.0)], 65.49 (t), 50.73 [t (d, J_{CF}=20.5)], 14.61 (q); IR: νₘₐₓ 3266, 2954, 2212, 1623, 1538, 1498, 1460, 1224, 1036 cm⁻¹; MS (ESI): m/z (%) 524 [MH⁺] (100); HR-MS (ESI) Calcd for C₂₅H₂₁N₇O₂F₂Cl: 524.1413, found 524.1404 (Δ−1.7 ppm).

### Example 7

The synthesis of quinoline 24 started from quinoline 19. Quinoline 19 was initially treated with the anion exchange resin AmberSep 900 OH to remove any traces of HCl. The ethoxy group was then removed by treating with BBr₃ in CH₂Cl₂ to give 7-hydroxyquinoline 20. This quinoline was treated with 1-fluoro-2-mesyloxy ethane (15) and K₂CO₃ in DMF to give 7-(2-fluoroethoxy)quinoline 22 in 75% yield from 19 and the acyl group was removed by heating in conc. HCl and water. The resulting 6-aminoquinoline 23 was linked to the Michael acceptor ester 4 using AlMe₃ mediated amidation in toluene. The Boc group was removed by treatment with HCl in 1,4-dioxane yielding quinoline 24 as the HCl salt in 67% yield (Scheme 6).

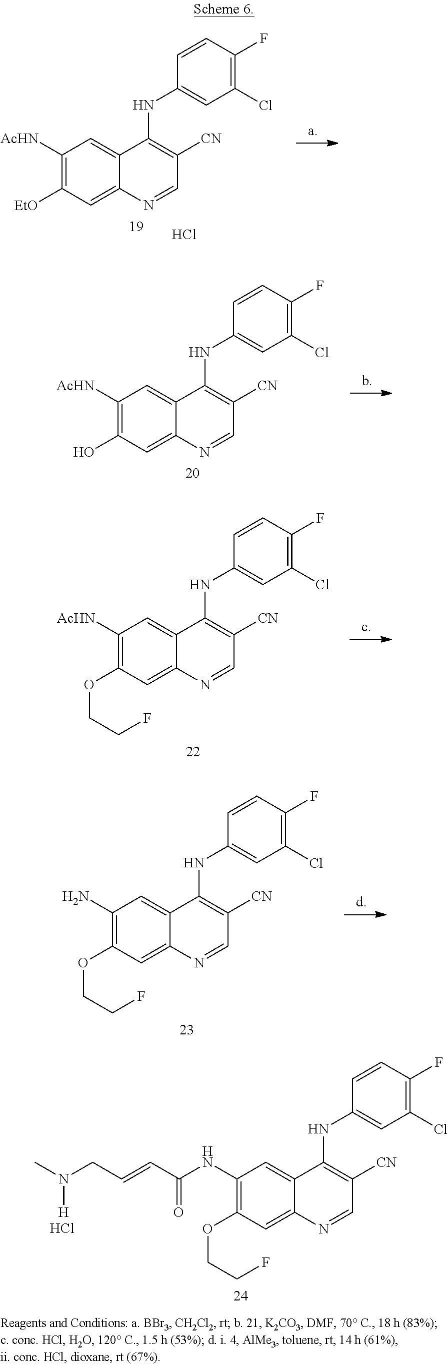

N-(4-(3-Chloro-4-fluorophenylamino)-3-cyano-7-hydroxyquinolin-6-yl]-acetamide (20): The quinoline 19 (500 mg, 1.15 mmol) was suspended in dry CH₂Cl₂ (50 mL) and BBr₃ (1.0 M in CH₂Cl₂, 5.7 mL, 5.7 mmol) was added dropwise at rt. The mixture was stirred 14 h and quenched with water (20 mL). The yellow precipitate was collected, washed with water (50 mL) and dried under vacuum. The title compound 20 was obtained as a yellow solid (166 mg, 40%) and used in the next step without further purification.

¹H NMR (400 MHz, MeOD) δ 9.12 (s, 1H), 8.79 (s, 1H), 7.68 (dd, J=1.5, 6.1, 1H), 7.51-7.45 (m, 1H), 7.41 (t, J=8.7, 1H), 7.35 (s, 1H), 2.28 (s, 3H); ¹³C NMR (101 MHz, MeOD) δ 170.9 (s), 158.2 [s, (d, J_{CF}=250.0)], 155.6 (s), 154.7 (s), 147.3 (d), 136.4 (s), 134.0 (s), 130.0 (s), 129.4 (d), 127.6 [d, (d, J_{CF}=7.9)], 121.4 [s, (d, J_{CF}=19.0)], 117.6 [d, (d, J_{GF}=22.8)], 113.7 (d), 113.1 (s), 111.4 (s), 102.3 (d), 86.1 (s), 22.7 (q); IR: νₘₐₓ 3357, 3018, 2925, 2228, 1613, 1539, 1495, 1469, 1238 cm⁻¹; MS (ESI): m/z (%) 371 [MH⁺] (100), 144 (25); HR-MS (ESI) Calcd for C₁₈H₁₃N₄O₂ClF: 371.0711, found 371.0707 (Δ−1.1 ppm).

N-R-(3-Chloro-4-fluorophenylamino)-3-cyano-7-(2-fluoro-ethoxy)-quinolin-6-yl]-acetamide (22), 6-Amino-4-(3-chloro-4-fluorophenylamino)-7-(2-fluoroethoxy)-quinoline-3-carbonitrile (23): The quinoline 20 (25 mg, 0.06 mmol) was heated with K₂CO₃ (41.4 mg, 0.30 mmol) and 2-mesyloxy-1-fluoroethane (21) (17 mg, 0.12 mmol) in DMF (1 mL) at 70° C. overnight. The mixture was cooled at rt, water (1 mL) was added and the quinoline 22 (yellow solid, 20 mg, 83%) was collected washed with water (3 mL) and diethyl ether (3 mL), dried under vacuum and used without further purification.

The yellow solid was refluxed in water (0.5 mL) and conc. HCl (0.5 mL) for 2.5 h, cooled at rt. The crude mixture was concentrated after reduced pressure, dissolved in water (2 mL) and neutralized with K₂CO₃. Quinoline 23 (8 mg, 53%) was collected as a yellow solid.

¹H NMR (400 MHz, MeOD) δ 8.33 (s, 1H), 7.94 (s, 1H), 7.47-7.00 (m, 4H), 5.09-4.74 (dm, J_{HF}=54.7, 2H), 4.48 (d, J_{HF}=28.5, 2H); ¹⁹F NMR (376 MHz, MeOD) δ−122.4, −225.1.

### General Procedure for the Synthesis of Compound Boc-24

The amino quinoline 23 (1 eq) and the Michael acceptor 4 (1.5 eq) were suspended and sonicated in dry CH₂Cl₂ or dry toluene (0.06 M) and AlMe₃ (2.0 M solution in hexane, 2 eq) was added dropwise at rt. The mixture was stirred at rt and monitored by TLC to the disappearance of the starting quinoline. The mixture was quenched with a saturated solution of NaHCO₃ and the phases were separated. The aqueous layer was extracted twice with CH₂Cl₂ and the combined organic layers washed with brine and dried over MgSO₄. The crude mixture was purification by chromatography on silica gel or plate silica gel TLC.

### General Procedure for the Synthesis of Compound 24, 25 and 28

Quinoline Boc-24, 11 or Boc-28 (1 eq) was dissolved in 1,4-dioxane (0.2 M) and conc. HCl (0.003 M) was added dropwise at rt. The mixture was stirred 5 min-1 h and concentrated. The precipitate was dissolved in MeOH, re-precipitated with diethyl ether, collected and dried under vacuum to give the title compounds as HCl salts.

(E)-4-Methylamino-but-2-enoic acid [4-(3-chloro-4-fluorophenylamino)-3-cyano-7-(2-fluoroethoxy)-quinolin-6-yl]-amide hydrochloride (24): yellow solid; 67% yield; ¹H NMR (400 MHz, MeOD) δ 9.27 (s, 1H), 8.93 (br s, 1H), 7.73 (dd, J=6.5, 2.5, 1H), 7.55-7.48 (m, 2H), 7.48-7.40 (m, 1H), 7.01 (dt, J=15.1, 6.5, 1H), 6.83 (d, J=15.4, 1H), 5.06-4.87 (dm, 2H), 4.67 (dm, J=28.7, 2H), 3.93 (d, J=6.5, 2H), 2.79 (s, 3H); ¹⁹F NMR (376 MHz, MeOD) δ−118.1, −223.9; ¹³C NMR (101 MHz, MeOD) δ 164.8 (s), 159.7 [s, (d, J_{CF}=255.2)], 156.9 (s), 156.2 (s), 149.55 (d), 138.6 (s), 135.5 (d), 135.3 (s), 131.2 (d), 131.0 (s), 130.8 (d), 128.9 [d, (d, J_{CF}=7.9)], 122.9 [s, (d, J_{CF}=19.3)], 118.6 [d, (d, J_{CF}=22.8)], 116.2 (d), 114.4 (s), 113.8 (s), 102.5 (d), 101.9 (s), 82.7 [t (d, J_{CF}=169.7)], 71.0 [t, (d, J_{CF}=19.8)], 50.2 (t), 33.3 (q); MS (ESI): m/z (%); 472 [MH⁺] (52), 257 (100); HR-MS (ESI) Calcd for C₂₃H₂₁N₅O₂ClF₂: 472.1354, found 472.1342 (Δ−2.1 ppm).

(E)-4-Prop-2-ynylaminobut-2-enoic acid [4-(3-chloro-4-fluorophenylamino)-3-cyano-7-ethoxy-quinolin-6-yl]-amide hydrochloride (25): yellow solid; 99% yield; ¹H NMR (400 MHz, d₆-DMSO) δ 11.02 (br s, 1H), 9.98 (s, 1H), 9.83 (s, 2H), 9.14 (s, 1H), 8.98 (s, 1H), 7.75 (d, J=6.6, 1H), 7.61-7.57 (m, 1H), 7.55 (t, J=8.5, 1H), 7.50-7.44 (m, 1H), 6.90-6.78 (m, 1H), 6.81 (t, J=11.9, 1H), 4.36 (q, J=7.0, 2H), 3.95 (s, 2H), 3.87 (d, J=5.4, 2H), 3.76 (t, J=2.3, 1H), 1.50 (t, J=6.9, 3H); ¹³C NMR (101 MHz, d₆-DMSO) δ 162.8 (s), 156.3 [s, (d, J_{CF}=247.0)], 155.2 (s), 153.2 (s), 149.3 (d), 139.6 (s), 135.4 (s), 134.3 (d), 129.6 (d), 128.6 (d), 127.9 (s), 126.7 [d (d, J_{CF}=7.0)], 119.9 [s (d, J_{CF}=18.9)], 117.4 [d, (d, J_{CF}=22.3)], 116.3 (d), 114.7 (s), 112.5 (s), 102.9 (d), 86.9 (s), 79.8 (d), 74.8 (s), 65.4 (t), 46.2 (t), 35.3 (t), 14.1 (q); MS (ESI): m/z (%) 478 [MH⁺] (100); HR-MS (ESI) Calcd for C₂₅H₂₂N₅O₂FCl: 478.1446, found 478.1448 (Δ0.4 ppm).

(E)-4-{(1-(3-Fluoro-4,5-dihydroxy-6-hydroxymethyl-tetrahydro-pyran-2-yl)-1H-[1,2,3]triazol-4-ylmethyl]-amino}-but-2-enoic acid [4-(3-chloro-4-fluorophenylamino)-3-cyano-7-ethoxy-quinolin-6-yl]-amide hydrochloride (28): yellow solid, 90% yield, ¹H NMR (500 MHz, D₂O) δ 8.88 (s, 1H), 8.64 (s, 1H), 8.41 (s, 1H), 7.56 (dd, J=2.2, 6.4, 1H), 7.38-7.31 (m, 2H), 7.25 (s, 1H), 6.85 (dt, J=6.7, 15.3, 1H), 6.58 (d, J=15.5, 1H), 6.06 (dd, J=2.6, 9.0, 1H), 4.87 (dt, J=50.6, 9.0, 1H), 4.47 (s, 2H), 4.33 (q, J=7.0, 2H), 4.03-3.94 (m, 3H), 3.85 (d, J=10.5, 1H), 3.77-3.64 (m, 2H), 3.60 (t, J=9.4, 1H), 1.47 (t, J=7.0, 3H); ¹³C NMR (126 MHz, D₂O) δ 164.7 (s), 160.5 [s, (d, J_{CF}=352.5),] 155.6 (s), 154.5 (s), 148.2 (d), 138.3 (s), 137.6 (s), 134.2 (d), 133.1 (s), 129.7 (d), 129.5 (d), 128.6 (s), 127.7 [d. (d, J_{CF}=8.1)], 126.0 (d), 117.5 [d, (d, J_{CF}=22.8)], 114.2 (d), 111.4 (s), 101.5 (d), 90.4 [d, (d, J_{CF}=187.5)], 85.6 (s), 84.7 [d, (d, J_{CF}=24.1)], 79.0 (s), 74.1 [d, (d, J_{CF}=16.5)], 68.5 [d, (d, J_{CF}=8.0)], 66.6 (t), 66.5 (d), 60.1 (t), 46.9 (t), 40.9 (t), 13.4 (q); HR-MS (ESI) Calcd for C₃₁H₃₂ClF₂N₅O₆: 685.2101, found 685.2109 (Δ1.2 ppm); MS (ESI): m/z (%) 685 [MH⁺] (20).

### Example 8

### Radiochemistry

Compound 25 was labelled by cycloaddition under Cu(I) catalysis by using [¹⁸]F-fluoroethyl azide [¹⁸F]-16 following a published procedure (Scheme 7) (Glaser, M., et al., Bioconjugate Chem. 2007, 18, 989-993; Smith, G. at al., J. Med. Chem. 2008, 51, 8057-8067; Glaser, et al., J. Label. Compd. Radiopharm. 2009, 52, 407-414).

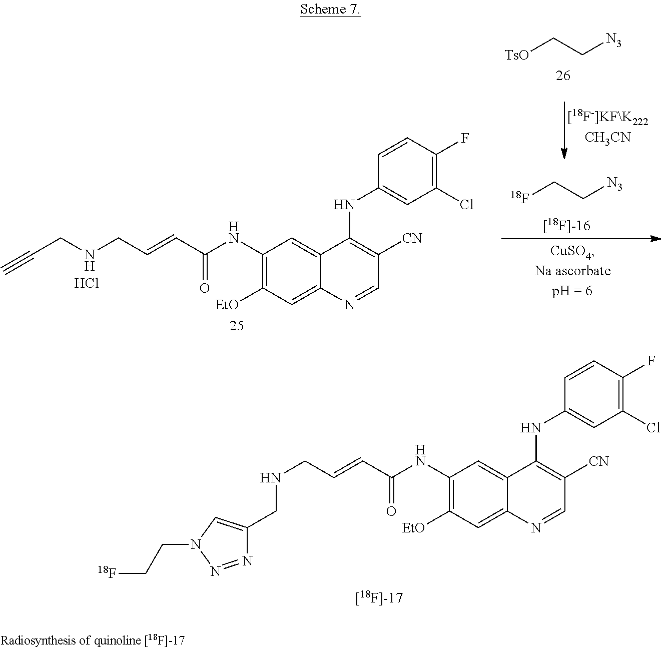 [SMILES: Cc1ccc(S(=O)(=O)OCCN=[N+]=[N-])cc1 ; C#CCNC/C=C/C(=O)Nc1cc2c(Nc3ccc(F)c(Cl)c3)c(C#N)cnc2cc1OCC ; Cl ; [N-]=[N+]=NCC[18F] ; CCOc1cc2ncc(C#N)c(Nc3ccc(F)c(Cl)c3)c2cc1NC(=O)/C=C/CNCc1cn(CC[18F])nn1]

[¹⁸]F-Fluoro ethyl azide [¹⁸F]-16 was synthesized from the corresponding tosyloxy ethyl azide 26 and [¹⁸F]KF/Kryptofix 222 at 80° C. for 15 min. The product was purified and collected by distillation; it was obtained with a 42.2±4.2% (n=12) decay corrected radiochemical yield. Precursor 25 was dissolved in MeCN/water, 1:1, mixed to the catalytic system and then heated with the azide [¹⁸F]-16 in MeCN at 80° C. for 15 min. The crude compound [¹⁸F]-17 was purified by semipreparative HPLC in a 37.0±3.6% (n=12) decay corrected radiochemical yield from azide [¹⁸F]-16 and >99% radiochemical purity. Compound [¹⁸F]-17 was formulated by solid-phase extraction with an efficiency of ˜90%. The identity of [¹⁸F]-17 was confirmed by co-elution with the non-radioactive compound and obtained with a specific activity of 6.8-0.2 GBq/μmol. The radioimaging agent [¹⁸F]-17 was stable for >4 h after formulation with PBS. The radiosynthesis including formulation took 3 h in total.

### General Procedure for the Synthesis of Compound [¹⁸F]17

Under an atmosphere of nitrogen, a buffered solution (sodium phosphate buffer, pH 6.0, 250 mM) of sodium ascorbate (50 μL, 8.7 mg, 43.2 μmol) was added to a Wheaton vial (3 mL) containing an aqueous solution of copper(II) sulfate (50 μL, 1.7 mg pentahydrate, 7.0 μmol). After one min, a solution of alkyne 25 (2.1 mg, 4.4 μmol) in MeCN/water, 1:1 (50 μL) was added followed by distilled [¹⁸F]-2-fluoroethylazide (94-740 MBq) in acetonitrile (100 μL). The mixture was heated at 80° C. for 15 min, the HPLC mobile phase [2]% MeCN (0.085% H₃PO₄), 500 μL] was added and the resulting mixture was purified by preparative radio-HPLC. The isolated HPLC fraction was diluted with water (5 mL) and loaded onto a SepPak C18-light cartridge (Waters) that had been conditioned with ethanol (5 mL) and water (10 mL). The cartridge was subsequently flushed with water (5 mL) and [¹⁸F]17 eluted with ethanol (0.1 mL fractions). The product fraction was diluted with PBS to provide an ethanol content of 10-20% (v/v).

### Example 9

### EGFR Tyrosine Kinase Enzyme Inhibition Assay

Assessment of the EGFR tyrosine kinase activity of control quinoline 1 together with quinolines 10, 13, 14, 17, 18, 24 and 25 was carried out using a cell free kinase activity inhibition assay as detailed below. BPDQ (4-N-(3-bromophenyl)quinazoline-4,6,7-triamine), a quinazoline based EGFR inhibitor was also included in this assay as a further reference standard. Concentrations of the compounds that inhibited EGFR kinase activity by 50% (IC₅₀) were calculated and are reported in Table 1.

TABLE 1
EGFR kinase activity inhibition profile for quinolines 1, 10, 13, 14, 17, 18, 24 and 25.

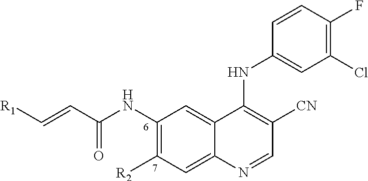

| | | | IC50 (nM) | | |
|---|---|---|---|---|---|
| | | | EGFR | | |
| | | | kinase | A431 EGFR | |
| | R₁ | R₂ | activity | autophosphorylation^{a} | LogP^{b} |
| BPDQ | | | 0.81 ± 0.01 | >1000 | 2.57 |
| 1 | CH₂NMe₂ | OEt | 0.24 ± 0.02 | 8.02 ± 0.75 | 4.18 |
| 10 | CH₂NHMe | OEt | 0.25 ± 0.06 | 5.35 ± 1.52 | 3.80 |
| 13 | CH₂N(Me)CH₂CH₂F | OEt | 0.80 ± 0.04 | 23.02 ± 12.0 | 4.38 |
| 14 | CH₂N(Me)-4-fluoro benzyl | OEt | 0.57 ± 0.12 | 16.52 ± 8.38 | 6.05 |
| 17 | 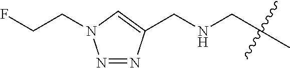 [SMILES: *CNCc1cn(CCF)nn1] | OEt | 1.81 ± 0.18 | 21.97 ± 9.06 | 3.85 |
| 18 | 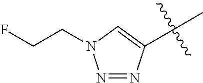 [SMILES: *c1cn(CCF)nn1] | OEt | 4.05 ± 0.57 | >1000 | 4.45 |
| 24 | CH₂NHMe | OCH₂CH₂F | 0.29 ± 0.03 | 8.12 ± 2.03 | 3.64 |
| 25 | CH≡CCH₂NHMe | OEt | 0.03 ± 0.01 | 60.2 ± 17.1 | 3.94 |

^{a}data are extracted from the concentration vs p-EGFR/total EGFR western blot absorbance ratio. Data are mean ± sem, n = 3 replicates.
^{b}LogP are calculated by ChemAxon's MarvinSketch, version 5.2.6.

All eight compounds inhibited EGFR kinase activity with IC₅₀ values in the low- or sub-nanomolar range which compares well with that of BPDQ (Table 1). Compound I appeared more potent than previously reported by Wissner (Wissner, A., et al., J. Med. Chem. 2003, 46, 49-63) presumably because of differences in the assay used. The same authors have shown that 1 functions as an irreversible inhibitor of EGFR. The IC₅₀ values are probably best interpreted in the context of reversible inhibition, as well as irreversible covalent binding resulting from interactions with the Michael acceptor (and other reactive) moieties. The sub-nanomolar kinase activity observed with compounds I and 10 was retained in compounds 13 and 14 demonstrating tolerance for small and large fluorine-containing substituents on the tertiary amine group. Fluorine substitution at the C-7 position was also tolerated as reflected in the comparably low IC₅₀ measured for compounds 10 and 24. Of interest to application of ‘click’ radiochemistry, substitution of fluoroethyl triazole on the Michael acceptor—exemplified by quinolines 17 and 18—was tolerated, with quinoline 17 being two-fold more active than 18. The activity of triazole derivatives 17 and 18 was reduced 10-20 fold relative to quinoline 1; the reason for this is unclear since previous modeling studies place the amine substituents at the edge of the kinase pocket. In addition, bulk substitution is accepted in the case of quinoline 14. Surprisingly the activity of the alkyne-containing quinoline 25 was in the picomolar range (30 pM) probably due to a previously undocumented π-π interaction that may also help to explain the high affinity of fluorobenzyl quinoline 14.

The inhibitory activity of quinolines 1, 10, 13, 14, 17, 18, 24 and 25 against EGFR kinase activity was measured by a time resolved fluorescence assay (DELFIA, Perkin-Elmer Life Sciences, Boston, Mass., USA). The compounds were dissolved in DMSO and diluted in DMSO to give final concentrations of 0.0001 to 100000 pg/mL. EGFR protein (E-3641, Sigma) was incubated with the compounds in a kinase buffer for 15 min at rt in accordance with manufacturer's instructions (DELFIA Tyrosine kinase kit; PerkinElmer). After 15 min at rt the kinase reaction was initiated by addition of 25 μM ATP, 25 mM MgCl₂, and 0.25 μM/L of biotinylated poly(Glu, Ala, Tyr) in 10 mM HEPES buffer, pH 7.4. The reaction proceeded at rt for 1 h and was stopped by addition of 100 mM EDTA. The enzyme reaction solution was diluted and aliquots added to 96-well ELISA streptavidin plates with shaking for 1 h. The plates were washed and phosphorylated Tyrosine was detected with Eu-labeled antiphosphotyrosine antibody (50 ng/well; PT66; PerkinElmer). After washing and enhancement steps, the plates were assessed in a Victor³ multi-label counter (PerkinElmer) using the EGFR Europium protocol. The concentration of compound that inhibited 50% of receptor phosphorylation activity (IC₅₀) was estimated by non-linear regression analysis using GraphPad Prism (Version 4.0 for Windows, GraphPad Software, San Diego Calif. USA).

### Example 10

### Cellular Activity and Lipophilicity

The ability of the compounds to be transported across cell membranes and to inhibit EGFR autophosphorylation was examined in highly EGFR-expressing A431 cells. Following the reversible binding protocol previously reported by Rabindran (Rabindran, S. K., et al., Cancer Res. 2004, 64, 3958-3965), the potency of the compounds to inhibit autophosphorylation of EGFR after 3 h of drug incubation (and a further 2 h of washing with drug free medium) was measured. Typical immunoblots demonstrating inhibition of EGFR autophosphorylation are shown in FIG. 1. In these studies the drug did not inhibit the expression of total EGFR protein. The inhibitory activity of compounds 1, 10, 13, 14, 17, and 24 on cellular EGFR autophosphorylation, (Table 1) translated well from that assessed in the cell-free system, with IC₅₀ values in the low nanomolar range. Interestingly, no cellular activity was apparent when dosing with quinoline 18. Furthermore, the cellular activity of quinoline 25 was in the low nanomolar range indicating that, although potent, the high affinity of this alkyne in the kinase assay did not directly translate into cellular activity.

The calculated Log P of the series ranged between 3.64 and 6.05 with fluorobenzyl substitution giving the highest Log P value. Log P provides an estimate of the compound's ability to pass through a cell membrane. Compounds with a Log P>5 are known to be nondruggable as defined by Lipinski's rule of 5 (Lipinski, C. A., et al., Adv. Drug. Deliv. Rev. 1997, 23, 3-25). The Log P of 14 being above the threshold may be sufficient to discard this compound at this stage. All the other compounds have a Log P>3 which suggests that no major difference could be drawn in terms of permeability among the different member of the library.

The ability of the compounds to diffuse into cells and to inhibit EGFR was assessed by measuring inhibition of receptor phosphorylation by quinolines 1, 10, 13, 14, 17, 18, 24 and 25 in A431 human epidermoid cancer cells (American Type Culture Collection, Manassas, Va., USA). The cells were maintained in DMEM (Sigma-Aldrich Company Ltd, Dorset, UK) supplemented with 10% fetal bovine serum (Lonza, UK), and 2 mM L-glutamine, 100 U/ml penicillin, 100 μg/mL streptomycin and 1 μg/mL fungizone (GIBCO) in 6 well plates incubated at 37° C. in a humidified incubator with 5% CO₂. The experiments were designed to assess irreversibility of EGFR inhibition by the compounds. Cells in exponential growth were incubated with quinolines 1, 10, 13, 14, 17, 18, 24 and 25 at various concentrations for 3 h. EGF (100 ng/ml) was added to the cells during the last 15 min to induce p-EGFR. The medium was removed and replaced with fresh compound-free medium for 1 h. The last step was then repeated twice. The cells were then washed with cold PBS and lysed in RIPA buffer (Invitrogen Ltd, Paisley, UK) supplemented with protease and phosphatase inhibitor cocktails (Sigma-Aldrich Company Ltd, Dorset, UK). Lysates were clarified by centrifugation. The following antibodies were used: rabbit polyclonal antibody anti-p-EGFR (Cell signalling Technology, Denver, Mass.; 1:1000) and rabbit polyclonal antibody anti-EGFR (Santa Cruz Biotechnology, Santa Cruz, Calif.; 1:1000) and mouse monoclonal antibody anti-β-actin (Abcam, UK; 1:10000) as primary antibodies. The secondary antibodies were Goat anti Rabbit IgG HRP (Santa Cruz Biotechnology Santa Cruz, Calif.; 1:2000) and Goat anti Mouse IgG HRP (Autogen Bioclear, UK; 1:2000). The same procedure was used to assess EGFR and phospho-EGFR expression in HCT116 human colon carcinoma cells.

### Example 11

### In Vitro Cell Uptake of Radiotracer [¹⁸F]17

Preliminary assessment of the suitability of [¹⁸F]17 as a candidate PET radioligand was carried out by incubation in A431 cells. The uptake of compound [¹⁸F]17 in the presence of verapamil, an inhibitor of the multi-drug resistant transporters, was studied. Uptake of [¹⁸F]17 was also modulated by pre-incubation with A431 cells of the natural ligand EGF and a 200 nM solution of quinoline 10 as a blocking agent. From FIG. 2 it is evident that addition of EGF increased the cellular uptake of compound [¹⁸F]17 by 22% and the pre-treatment with compound 10 decreased cellular uptake by 30%. In a separate study, also shown in FIG. 2, comparative uptake of [¹⁸F]17 was carried out in A431 and in the non EGFR overexpressing MCF-7 breast carcinoma cell line. Uptake of [¹⁸F]17 was two fold higher in the EGFR overexpressing A431 cell line relative to MCF-7 cells. Experiments reported in FIG. 2 demonstrated an EGFR dependant uptake of [¹⁸F]17.

Cells were cultured in 6-well plates (n=3) in full growth medium until they reached approximately 80% confluence. The cells were cultured in serum free medium 24 h and 100 ng/mL EGF or corresponding vehicle was added 15 min before [¹⁸F]17 incubation. For one set of studies, the cells were also incubated with 200 nM quinoline 10 for 13 min prior to addition of radiotracer. Furthermore, all cells were pre-treated with 100 μM verapamil 5 min prior the addition of radiotracer [¹⁸F]17. Radiotracer [¹⁸F]17 was added to each well (˜0.37 MBq in 100 μL; ˜15 GBq/μmol specific radioactivity) and incubated for 1 h at 37° C. The cells were washed 3 times with ice-cold PBS and lyses in RIPA buffer. Aliquots of the lysates were transferred in counting tubes and fluorine-18 radioactivity was immediately determined using a Packard Cobra II gamma counter (PerkinElmer, UK). BCA Protein assay (Pierce, UK) was performed for all samples and data are normalized and expressed as counts/mg of protein.

### Example 12

### Biodistribution, Metabolic Stability and Initial PET Imaging

The 60 minutes tissue biodistribution of [¹⁸F]17 in A431 tumor bearing mice, expressed as tissue to blood ratios, is shown in FIG. 3. The radiotracer appears to be eliminated via both the hepatobiliary and renal routes as the highest tissue radioactivities were found in gallbladder and urine. Elimination of the radiotracer into the gut may also account for the high radioactivity in early part of the intestine. Low radioactivity was observed in most other organs including muscle and heart. The low uptake in bone suggests that the radiotracer does not undergo defluorination. Tumor (A431 xenograft) uptake was approximately four-fold higher than that of muscle (FIG. 3).

The in vivo metabolic stability in normal mice using liver and plasma extracts at 2, 30 and 60 minutes post-injection was investigated. Sample analysis was accomplished by radio-HPLC. Typical radiochromatograms are shown in FIG. 4. At 2 min, only parent compound was observed in plasma. A more polar radioactive metabolite was seen in plasma at 30 min and similarly one low level metabolite peak was seen in liver. The metabolic stability data, summarized in Table 2, demonstrates that parent radiotracer [¹⁸F]17 remains a major component of both liver and plasma even at 60 minutes post injection; indicative of a good stability in vivo.

TABLE 2
In vivo metabolism of compound [¹⁸F]17 at
selected time points, showing the proportion of
compound [¹⁸F]17 present in plasma and liver extracts^{a.}

| Time (min) | Parent (Liver) | Parent (Plasma) |
|---|---|---|
| 2 | 95.00 ± 1.00 | 98.92 ± 1.06 |
| 30 | 85.06 ± 1.75 | 62.81 ± 1.70 |
| 60 | 75.45 ± 2.73 | 49.75 ± 6.27 |

^{a}The extracts were analyzed by radio-HPLC [50% MeCN (0.085% H₃PO₄)]. The values are the average of 3 independent studies per time point. Proportion of compound [¹⁸F]17 in plasma and liver were calculated by comparison of compound [¹⁸F]17 peak to total radioactivity present on chromatogram. The efficiency of the extraction from plasma was 83.5%.

The potency of compound [¹⁸F]17 to detect an A431 xenograft by small animal PET imaging was further assessed. A summed image for a dynamic PET scan 30-60 minutes post-injection of [¹⁸F]17 is shown in FIG. 5. Tumor uptake is clearly observable; significant radiotracer localization was also seen in the abdominal area.

### In Vivo PET Imaging and Biodistribution

A431 and HCT116 xenografts were established by s.c. injection of 5×10⁶ cells on the back of 6- to 8-week-old female nu/nu Balb/c mice (Harlan). All animal work was performed by licensed investigators in accordance with the United Kingdom's “Guidance on the Operation of Animals (Scientific Procedures) Act 1986” (HMSO, London, United Kingdom, 1990) (Workman, P.; Aboagye, E. O.; Balkwill, F.; Balmain, A.; Bruder, G.; Chaplin, D. J.; Double, J. A.; Everitt, J.; Farningham, D. A. H.; Glennie, M. J.; Kelland, L. R.; Robinson, V.; Stratford, I. J.; Tozer, G. M.; Watson, S.; Wedge, S. R.; Eccles, S. A. Br. J. Cancer., 102, 1555-1577). When tumours reached ˜100 mm³, animals (n=3) were scanned on a dedicated small animal CT/PET scanner (Siemens Multimodality Inveon, Siemend Molecular Imaging Inc., Knoxyille, USA) following a bolus i.v. injection of 3.7 MBq of [¹⁸F]17. Dynamic emission scans were acquired in list-mode format over 60 minutes. Cumulative images of the dynamic data (30 to 60 min) were iteratively reconstructed (OSEM3D) and used for visualization of radiotracer uptake to define the regions of interest (ROIs) with the Siemens Inveon Research Workplace software (three-dimensional ROIs were defined for each tumour). The count densities (counts/mL) were averaged for all ROIs at each of the 19 time points to obtain a time versus radioactivity 950 curve (TAC). Tumour TACs were normalized to that total counts within the whole body at each of the time points to obtain the normalized uptake value expressed as % ID/mL. Direct [¹⁸F]17 tissue biodistribution was assessed subsequent to the PET scan. For this, mice were sacrificed by exsanguination via cardiac puncture under general anesthesia (isofluorane inhalation) and tissues were excised, weighted and immediately counted for fluorine-18 radioactivity on a Cobra II Auto-Gamma counter (Packard Instruments, Meriden, CTA). Data were expressed as tissue to blood ratios and % injected dose per gram (% ID/g).

### Metabolism Studies

Non-tumor-bearing mice were injected intravenously with 3.7 MBq of radiotracer [¹⁸F]17. Plasma and liver were collected at the indicated time and were snap-frozen in liquid nitrogen for subsequent HPLC analysis. For extraction, ice cold MeOH (1.5 mL) was added to plasma. The mixture was centrifuged (15493 g, 4° C., 3 min) and the resulting supernatant was evaporated to dryness under vacuum at 40° C. using a rotary evaporator. Liver samples were homogenized with ice cold MeOH (1.5 mL) using an IKA Ultra-Turrax T-25 homogenizer prior to centrifugation. The supernatant was then decanted and evaporated to dryness. The samples were re-suspended in HPLC mobile phase (1.2 mL) and filtered through a Whatman PTFE syringe filter (0.2 μm). The samples (1 mL) were analyzed by radio-HPLC on an Agilent 1100 series HPLC system (Agilent Technologies, Stockport, UK) equipped with a γ-RAM model 3 gamma-detector (IN/US Systems Inc., Florida) and the Laura 3 software.

The stationary phase comprised of a Waters μBondapak C18 reverse-phase column (300 mm×7.8 mm) by using a mobile phase comprising of water (0.085% H₃PO₄)/acetonitrile (0.085% H₃PO₄) (50:50) running in isocratic mode at a flowrate of 3 mL/min.

### Extraction Efficiency for Plasma

To pre-weighed counting tubes (n=4) Dulbecco's phosphate buffered saline (Sigma, Gillingham, UK) (200 μL) was added and to a further set of tubes (n=4) mouse plasma extract (Mouse plasma lithium heparin-CD-1-Mixed Gender, pooled, Sera Laboratories International, West Sussex, UK) (200 μL) was added and the samples stored on ice until radiopharmaceutical addition. Formulated radiotracer [¹⁸F]17 was added to a set (n=4) of blank counting tubes and to the tubes containing PBS or plasma. The samples were then incubated at 37° C. for 30 minutes and then snap frozen using dry ice. Immediately prior to extraction samples were thawed on ice and ice-cold methanol (1.5 mL) added. The samples were then centrifuged (15493×g, 4° C., 3 minutes). The supernatant was then decanted and evaporated to dryness. The sample was then re-suspended in HPLC mobile phase (1.1 mL) and filtered (Whatman PTFE 0.2 μm, 13 mm filters). Total radioactivity for each sample (control—100%—, PBS extract—95.4%—and plasma extract—83.5%—) was then measured on a Cobra-II Gamma Counter.

### Example 13

### Selectivity of [¹⁸F]17 (High EGFR Expressing A431 Xenografts vs Low EGFR Expressing HCT116 Xenografts)

We further assessed the potency of compound [¹⁸F]17 to detect high EGFR expressing A431 xenografts relative to low EGFR expressing HCT116 xenografts by small animal PET imaging. PET images from representative A431 and HCT116 tumour bearing mice with [¹⁸F]17 (FIG. 6) demonstrated localization and visualization of the tumour, particularly in A431. The time activity curves (TACs) of A431 and HCT116 tumours extracted from the PET data (FIG. 6) showed more rapid washout of the radiotracer from HCT116 tumours. Significant radiotracer localization was also seen in the abdominal region consistent with the biodistribution data. The PET data were corroborated by ex vivo tumour uptake and western blot analysis of EGFR protein content of the two tumour types (FIG. 6). There was a fourfold higher uptake in A431 xenografts compared to HCT116 xenografts (Pisaneschi, F.; Nguyen, Q.-D.; Shamsaei, E.; Glaser, M.; Robins, E.; Kaliszczak, M.; Smith, G.; Spivey, A. C.; Aboagye, E. O. Bioorg. Med. Chem., 18, 6634-6645.)

1. A compound of formula I, 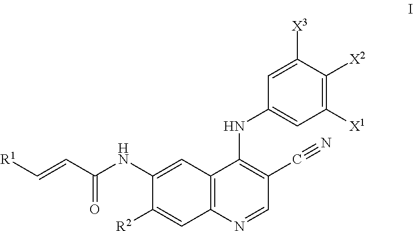
wherein:
R¹ represents Het^{a} or a C₁₋₃₀ alkyl group optionally substituted by one or more A groups;
R² represents a C₁₋₃₀ alkyl group optionally substituted by one or more B groups or one or more halogen atoms; a C₁₋₁₂-alkoxy group optionally substituted by one or more halogen atoms or hydroxyl groups; or Het^{b};
X¹ and X³ each independently represents hydrogen or a halogen;
A represents Het^{c}, —N(R^{a1})R^{a2}, —OR^{a3} or —SR^{a4};
B represents —N(R^{b1})R^{b2}, —OR^{b3} or —SR^{b4};
X² represents hydrogen, a halogen, OR^{c1}, SR^{c2} or a C₁₋₃₀ alkyl group optionally substituted by one or more halogen atoms or one or more C groups;
C represents —N(R^{d1})R^{d2}, —OR^{d3} or —SR^{d4};
Het^{a} represents a heteroaryl group which may be optionally substituted by one or more halogen atoms or R^{d} groups;
Het^{b} represents a heteroaryl group which may be optionally substituted by one or more halogen atoms or R^{e} groups;
Het^{c} represents a heteroaryl group which may be optionally substituted by one or more halogen atoms or R^{f} groups;
R^{a1} to R^{a4}, R^{b1} to R^{b4} and R^{d1} to R^{d4} each independently represent hydrogen, a C(O)OR^{g} group, a C₁₋₆ alkyl group or a —C(O)—C₁₋₆ alkyl group, which latter two groups are optionally substituted with one or more D groups, one or more E groups and/or one or more halogen atoms;
R^{c1} and R^{c2} independently represent a C₁₋₁₂ alkyl group, a C₁₋₄-alkyl-C₃₋₈-cycloalkyl group, a C₁₋₄-alkyl-aryl group or a C₁₋₄-alkyl-Het^{d} group;
D represents an aryl group optionally substituted by one or more halogen atoms or R^{h} groups, or a Het^{e} group;
Het^{d} represents a heteroaryl group which may be optionally substituted by one or more halogen atoms or Rⁱ groups;
Het^{e} represents a heteroaryl group which may be optionally substituted by one or more halogen atoms or R^{j} groups;
E represents —O—N(R^{k})R^{l} or —O—N═C(R^{m})Rⁿ;
R^{d}, R^{e}, R^{f}, R^{g}, R^{h}, Rⁱ and R^{j} independently represent:
a C₁₋₆ alkyl group optionally substituted by one or more halogen atoms or another suitable leaving group (e.g. a p-toluenesulfonate, a methanesulfonate, a p-nitrobenzenesulfonate, an o-nitrobenzenesulfonate or a trifluoromethanesulfonate group); or
a Q group 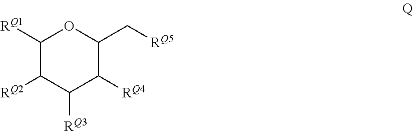
wherein one of R^{Q1} to R^{Q5} represents the point of attachment to the quinoline-containing portion of the molecule, one or more of R^{Q1} to R^{Q5} represents a halogen atom or another suitable leaving group (e.g. a p-toluenesulfonate, a methanesulfonate, a p-nitrobenzenesulfonate, an o-nitrobenzenesulfonate or a trifluoromethanesulfonate group), and the remaining R^{Q1} to R^{Q5} groups represent —OH;
R^{k}, R^{l}, R^{m} and Rⁿ each independently represent hydrogen or a C₁₋₁₂ alkyl group optionally substituted by one or more halogen atoms, —OR^{o} or —N(R^{p})R^{q} groups;
R^{o}, R^{p} and R^{q} each independently represent hydrogen or a C₁₋₄ alkyl group;
or a pharmaceutically-acceptable salt thereof,
provided that
(i) when X³ represents hydrogen, X² represents fluoro, and X¹ represents chloro,
(a) when R² represents —O—CH₂CH₃, R¹ does not represent —CH₂—N(CH₃)₂ or —CH₂—N(H)CH₃,
(b) when R² represents —O—CH₃, R¹ does not represent —CH₂—N(CH₃)₂, —CH₂—N(CH₂CH₃)₂, —CH(CH₃)—N(CH₃)₂ and —CH(CH₃)—N(CH₂CH₃)₂;
(c) when R² represents —O—CF₃, R¹ does not represent —CH₂—N(CH₃)₂;
(ii) when X² and X³ represent hydrogen, X¹ represents bromo, and R¹ represents —CH₂—N(CH₃)₂, R² does not represent —O—CH₃ or —O—CH₂CH₃;
(iii) when X¹ represents chloro, X³ represent hydrogen, R¹ represents —CH₂—N(CH₃)₂ and R² represents —O—CH₃, X² does not represent imidazol-1-yl; and
(iv) when R² represents —O—CH₂CH₃ or —O—CH₃, X¹ represents hydrogen or chlorine, X³ represents hydrogen or chlorine and X² represents OR^{c1}, the compound contains at least one fluorine atom.

2. A compound as claimed in claim 1, wherein R¹ represents Het^{a} or a C₁₋₆ alkyl group optionally substituted by one or more A groups.

3. A compound as claimed in claim 1, wherein A represents —N(R^{a1})R^{a2}.

4. A compound as claimed in claim 1, wherein R¹ represents —CH₂N(CH₃)CH₂CH₂F, —CH₂N(CH₃)CH₂C₆H₄F, —CH₂NH(CH₃), —CH₂NHCH₂C≡CH, —CH₂N(boc)CH₂C≡CH, —C≡CH, —CH₂NHCH₂CH₂ONH₂, —CH₂NHC(O)CH₂ONH₂

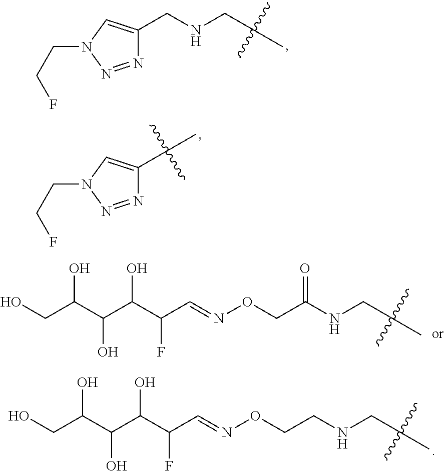

5. A compound as claimed in claim 1, wherein R² represents a C₁₋₆-alkoxy group optionally substituted by one or more halogen atoms, or Het^{b}.

6. A compound as claimed in claim 1, wherein R² represents —OCH₂CH₃, —OCH₂CH₂F, —C≡CH or

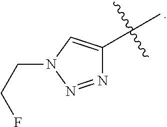 [SMILES: *c1cn(CCF)nn1]

7. A compound as claimed in claim 1, wherein X² represents a halogen, OR^{c1} or SR^{c2}.

8. A compound as claimed in claim 1, wherein X¹ and X³ independently represent hydrogen or chlorine and X² represents fluorine,

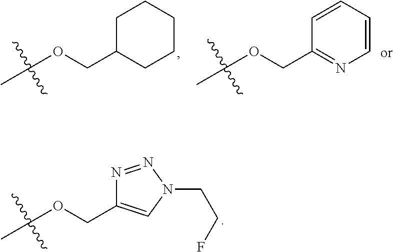 [SMILES: *OCC1CCCCC1 ; *OCc1ccccn1 ; *OCc1cn(CCF)nn1]

9. A compound as claimed in claim 1, which is selected from the group:
{(E)-3-[4-(3-Chloro-4-fluoro-phenylamino)-3-cyano-7-ethoxy-quinolin-6-ylcarbamoyl]-allyl}-prop-2-ynyl-carbamic acid tert-butyl ester;
(E)-Pent-2-en-4-ynoic acid [4-(3-chloro-4-fluorophenylamino)-3-cyano-7-ethoxy-quinolin-6-yl]-amide;
(E)-4-[(2-Fluoroethyl)methyl amino]-but-2-enoic acid [4-(3-chloro-4-fluorophenylamino)-3-cyano-7-ethoxyquinoline-6-yl]amide;
(E)-4-[(4-Fluorobenzyl)methylamino]-but-2-enoic acid [4-(3-chloro-4-fluorophenylamino)-3-cyano-7-ethoxyquinoline-6-yl]amide;
(E)-4-{[1-(2-Fluoro-ethyl)-1H-[1,2,3]triazol-4-ylmethyl]-amino}-but-2-enoic acid [4-(3-chloro-4-fluoro-phenylamino)-3-cyano-7-ethoxy-quinolin-6-yl]-amide hydrochloride;
(E)-N-[4-(3-Chloro-4-fluorophenylamino)-3-cyano-7-ethoxyquinolin-6-yl]-3-[1-(2-fluoro-ethyl)-1H-[1,2,3]triazol-4-yl]-acrylamide;
(E)-4-Methylamino-but-2-enoic acid [4-(3-chloro-4-fluorophenylamino)-3-cyano-7-(2-fluoroethoxy)-quinolin-6-yl]-amide hydrochloride;
(E)-4-Prop-2-ynylaminobut-2-enoic acid [4-(3-chloro-4-fluorophenylamino)-3-cyano-7-ethoxy-quinolin-6-yl]-amide hydrochloride;
(E)-4-Methylamino-but-2-enoic acid {4-(3-chloro-4-fluoro-phenylamino)-3-cyano-7-[1-(2-fluoro-ethyl)-1H-[1,2,3]triazol-4-yl]-quinolin-6-yl}-amide;
Toluene-4-sulfonic acid 2-[4-({(E)-3-[4-(3-chloro-4-fluoro-phenylamino)-3-cyano-7-ethoxy-quinolin-6-ylcarbamoyl]-allylamino}-methyl)-[1,2,3]triazol-1-yl]-ethyl ester;
(E)-4-{[1-(2-Fluoro-ethyl)-1H-[1,2,3]triazol-4-ylmethyl]-amino}-but-2-enoic acid [4-(3-chloro-4-(cyclohexylmethoxy)-phenylamino)-3-cyano-7-ethoxy-quinolin-6-yl]-amide;
(E)-4-{[1-(2-Fluoro-ethyl)-1H-[1,2,3]triazol-4-ylmethyl]-amino}-but-2-enoic acid [4-(3-chloro-4-((pyridin-2-yl)methoxy)-phenylamino)-3-cyano-7-ethoxy-quinolin-6-yl]-amide;
(E)-4-{Methylamino}-but-2-enoic acid [4-(3-chloro-4-((1-(2-fluoro-ethyl)-1H-[1,2,3]triazol-4-yl)methoxy)-phenylamino)-3-cyano-7-ethoxy-quinolin-6-yl]-amide;
(E)-4-{2-(aminooxy)-ethylamino}-but-2-enoic acid [4-(3-chloro-4-fluoro-phenylamino)-3-cyano-7-ethoxy-quinolin-6-yl]-amide;
(E)-4-{2-[2-Fluoro-3,4,5,6-tetrahydroxy-hex-(E)-ylideneaminooxy]-ethylamino}-but-2-enoic acid [4-(3-chloro-4-fluoro-phenylamino)-3-cyano-7-ethoxy-quinolin-6-yl]-amide;
(E)-4-{2-[2-Fluoro-3,4,5,6-tetrahydroxy-hex-(E)-ylideneaminooxy]-acetylamino}-but-2-enoic acid
[4-(3-chloro-4-fluoro-phenylamino)-3-cyano-7-ethoxy-quinolin-6-yl]-amide;
(E)-4-{[1-(3-Fluoro-4,5-dihydroxy-6-hydroxymethyl-tetrahydropyran-2-yl)-1H-[1,2,3]triazol-4-ylmethyl]-amino}-but-2-enoic acid [4-(3-chloro-4-fluoro-phenylamino)-3-cyano-7-ethoxyquinolin-6-yl]-amide; and
(E)-4-[(2-Fluoroethyl)-methyl-amino]-but-2-enoic acid [4-(3-chloro-4-fluorophenylamino)-3-cyano-7-(2,3-dihydroxypropoxy)-quinolin-6-yl]-amide.

10. A compound as defined in claim 1, but not limited by the provisos, comprising a positron emitting radioisotope, a single photon emitting radioisotope and/or another radioisotope.

11. A compound as claimed in claim 10, comprising a positron emitting radioisotope which is [¹⁸F].

12. A compound as claimed in claim 10, comprising a positron and/or single photon emitting radioisotope selected from 11C, ⁶¹Cu, ⁶⁴Cu, ⁶⁷Cu, ⁶⁷Ga, ⁶⁸Ga, ⁷⁵Br, ⁷⁶Br, ^{94m}Tc, ^{99m}Tc, ¹¹¹In, ¹²³I, ¹²⁴I, ¹²⁵I, ¹³¹I, and ²⁰¹Tl, and/or another radioisotope which is ³H, ¹⁴C or ³⁵S.

13. A compound as claimed in claim 10 for use as a positron emission tomography (PET) imaging agent.

14. A compound as claimed in claim 1 for use in the inhibition of epidermal growth factor receptor tyrosine kinase activity or the inhibition of HER2 activity.

15. A compound as claimed in claim 1 for use in the treatment of cancer.

16. A pharmaceutical formulation including a compound of formula I, as claimed in claim 1, or a pharmaceutically acceptable salt thereof, in admixture with a pharmaceutically acceptable adjuvant, diluent or carrier.

17. (canceled)

18. (canceled)

19. A positron emission tomography (PET) imaging agent comprising the compound of formula I, as claimed in claim 10 or a pharmaceutically acceptable salt thereof.

20. A method of treating or preventing a disease in which inhibition of epidermal growth factor receptor tyrosine kinase activity or the inhibition of HER2 activity is desired and/or required, which method comprises administering a therapeutically effective amount of a compound of formula I as claimed in claim 1 or a pharmaceutically-acceptable salt thereof to a patient in need thereof.

21. A method of treating or preventing cancer, which method comprises administering a therapeutically effective amount of a compound of formula I as defined in claim 1 or a pharmaceutically-acceptable salt thereof to a patient in need thereof.

22. A combination product which comprises a pharmaceutical formulation including a compound of formula I as defined in claim 1 but not limited by the provisos or a pharmaceutically-acceptable salt thereof an ABC transporter inhibitor, and a pharmaceutically-acceptable adjuvant, diluent or carrier.

23. A combination product as claimed in claim 22 which comprises a kit of parts comprising components:
(a) a pharmaceutical formulation including a compound of formula I but not limited by the provisos or a pharmaceutically-acceptable salt thereof in admixture with a pharmaceutically-acceptable adjuvant, diluent or carrier; and
(b) a pharmaceutical formulation including an ABC transporter inhibitor in a mixture with a pharmaceutically-acceptable adjuvant, diluent or carrier,
wherein components (a) and (b) are each provided in a form that is suitable for administration in conjunction with the other.

24. A process for the preparation of a compound of formula I as claimed in claim 1, which comprises:
(i) for compounds of formula I in which R² represents a C₁₋₁₂-alkoxy group substituted by one or more halogen atoms, reacting a compound of formula II, 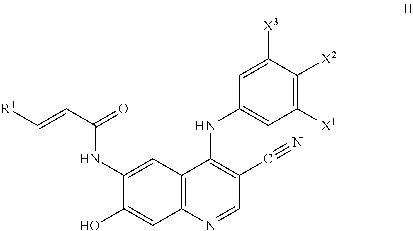
or a protected derivative thereof, wherein R¹, X¹, X² and X³ are as defined in claim 1 with a compound of formula III,
R^{2a}-L¹ III
wherein R^{2a} represents the optionally substituted C₁₋₁₂ alkyl portion of R², and L¹ represents a suitable leaving group; or
(ii) for compounds of formula I in which R¹ represents an optionally substituted 1,2,3-triazole group, reacting a compound of formula IV, 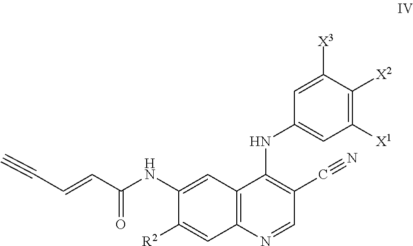
wherein R², X¹, X² and X³ are as defined in claim 1, with a compound of formula V,
R^{1d}—N₃ V
wherein R^{1d} represents H or R^{d} as defined in claim 1; or
(iii) reacting a compound of formula VI, 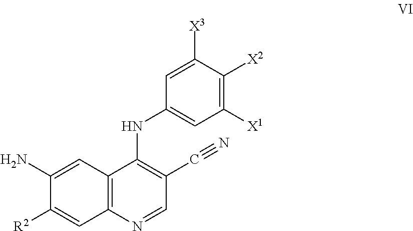
wherein R², X¹, X² and X³ are as defined in claim 1, with a compound of formula VII, 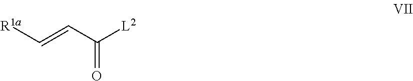
wherein R^{1a} represents R¹ as defined in claim 1, and L² represents a suitable leaving group; or
(iv) for compounds in which R¹ represents a C₁₋₃₀ alkyl group substituted by one or more —N(R^{a1})R^{a2} groups wherein at least one of R^{a1} and R^{a2} is a —CH₂—R^{ax} group wherein R^{ax} represents a D group, an E group, a halogen or a C₁₋₅ alkyl group optionally substituted with one or more D groups, one or more E groups and/or one or more halogen atoms, reacting a compound of formula VIII, 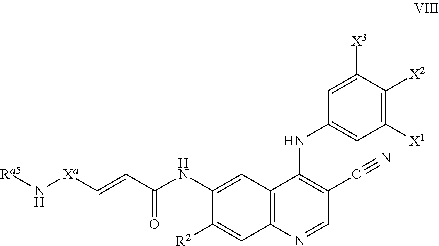
wherein R², X¹, X² and X³ are as defined in claim 1, R^{a5} represents either R^{a1} or R^{a2}, and X^{a} represents the optionally substituted C₁₋₃₀ alkyl group of R¹, with a compound of formula IX, 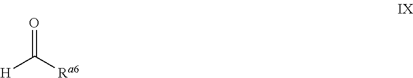
wherein R^{a6} represents R^{ax} as defined above, followed by reduction; or
(v) for compounds of formula I in which R¹ represents a C₁₋₃₀ alkyl group optionally substituted by —N(R^{a1})R^{a2}, reacting a compound of formula X, 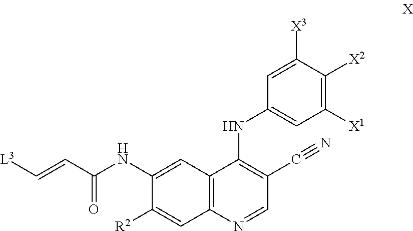
wherein R², X¹, X² and X³ are as defined in claim 1, and L³ represents a leaving group, with a compound of formula XI,
NH(R^{a1′})R^{a2′} XI
wherein R^{a1′} and R^{a2′} represent R^{ai} and R^{a2} as defined in claim 1, respectively; or
(vi) for compounds of formula I wherein one or more of R^{d}, R^{e}, R^{f}, R^{g}, R^{h}, Rⁱ and R^{j} represents a C₁₋₆ alkyl group substituted by one or more halogen atoms, reaction of a compound of formula I wherein the corresponding R^{d}, R^{e}, R^{f}, R^{g}, R^{h}, Rⁱ or R^{j} group represents a C₁₋₆ alkyl group substituted by one or more leaving groups, with an appropriate metal halide.

25. A process for the preparation of a pharmaceutical formulation as defined in claim 16, which process comprises bringing into association a compound of formula I, or a pharmaceutically acceptable salt thereof and a pharmaceutically-acceptable adjuvant, diluent or carrier.

26. A process for the preparation of a combination product as defined in claim 22, which process comprises bringing into association a compound of formula I, but not limited by the provisos or a pharmaceutically acceptable salt thereof and an ABC transporter inhibitor, and at least one pharmaceutically-acceptable adjuvant, diluent or carrier.
